SEQUENCE LISTING (15 sequences)

SEQ ID NO: 1 - protein, 209 aa, Homo sapiens
ADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 297 aa, Homo sapiens
SDIEEVVEEYEEEEQEEAAVEEEEDWREDEDEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 3 - protein, 137 aa, Homo sapiens
TALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 4 - protein, 164 aa, Homo sapiens
EMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 5 - protein, 744 aa, Homo sapiens
GVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 6 - protein, 260 aa, Homo sapiens
WVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 7 - protein, 133 aa, Homo sapiens
DPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 8 - protein, 165 aa, Homo sapiens
APKKAKKRAGGANSNVFSMFEQTQIQEFKEAFTIMDQNRDGFIDKNDLRDTFAALGRVNVKNEEIDEMIKEAPGPINFTVFLTMFGEKLKGADPEETILNAFKVFDPEGKGVLKADYVREMLTTQAERFSKEEVDQMFAAFPPDVTGNLDYKNLVHIITHGEEKD

SEQ ID NO: 9 - protein, 1938 aa, Homo sapiens
TDAQMADFGAAAQYLRKSEKERLEAQTRPFDIRTECFVPDDKEEFVKAKILSREGGKVIAETENGKTVTVKEDQVLQQNPPKFDKIQDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQALALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMEADLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 10 - protein, 1934 aa, Homo sapiens
GDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 11 - protein, 15 aa, Homo sapiens
SSDAAREPRPAPAPI

SEQ ID NO: 12 - protein, 15 aa, Homo sapiens
VDEERYDIEAKVTKN

SEQ ID NO: 13 - protein, 15 aa, Homo sapiens
DIEAKVTKNITEIAD

SEQ ID NO: 14 - protein, 15 aa, Homo sapiens
LDLRAHLKQVKKEDT

SEQ ID NO: 15 - protein, 14 aa, Homo sapiens
ALSGMEGRKKKFES

(19) United States
(12) Patent Application Publication
(10) Pub. No.: US 2011/0136141 A1
(43) Pub. Date: Jun. 9, 2011

(54) PEPTIDE REAGENTS AND METHOD FOR INHIBITING AUTOANTIBODY ANTIGEN BINDING

(75) Inventors: Maciej Adamczyk, Gurnee, IL (US); Jefffrey R. Brashear, Mundelein, IL (US); Phillip G. Mattingly, Third Lake, IL (US)

(73) Assignee: Abbott Laboratories, Abbott Park, IL (US)

(21) Appl. No.: 12/630,671

(22) Filed: Dec. 3, 2009

(51) Int. Cl. G01N 33/53; G01N 33/566

(52) U.S. Cl. 435/7.9; 435/7.1; 436/501

(57) ABSTRACT

The present disclosure provides immunoassays and kits for detection or quantification of an protein of interest in a test sample that potentially contains endogenously produced autoantibodies reactive with the analyte.

### RELATED APPLICATION INFORMATION

None.

### INCORPORATION OF SEQUENCE LISTING

The entire contents of a paper copy of the “Sequence Listing” and a computer readable form of the sequence listing on diskette, containing the file named 400797_SequenceListing_ST25.txt, which is 56 kilobytes in size and was created on Dec. 3, 2009, 2009, are herein incorporated by reference.

### TECHNICAL FIELD

The present disclosure relates to methods and kits for detecting a protein of interest in a test sample, and in particular to methods and kits for detecting the protein in a human test sample that may contain endogenous anti-analyte antibodies.

### BACKGROUND

Immunoassay techniques have been known for the last few decades and are now commonly used in medicine for a wide variety of diagnostic purposes to detect target analytes in a biological sample. Immunoassays exploit the highly specific binding of an antibody to its corresponding antigen, wherein the antigen is the target analyte. Typically, quantification of either the antibody or antigen is achieved through some form of labeling such as radio- or fluorescence-labeling. Sandwich immunoassays involve binding the target analyte in the sample to the antibody site (which is frequently bound to a solid support), binding labeled antibody to the captured analyte, and then measuring the amount of bound labeled antibody, wherein the label generates a signal proportional to the concentration of the target analyte inasmuch as labeled antibody does not bind unless the analyte is present in the sample.

A problem with this general approach is that many patients have circulating endogenous antibodies, or “autoantibodies” against an analyte of clinical interest. For example, autoantibodies have been described for cardiac troponin, myeloperoxidase (MPO), prostate specific antigen (PSA), and thyroid stimulating hormone (TSH), and other clinically significant analytes. Autoantibodies create interference in typical sandwich immunoassays that are composed of two or more analyte-specific antibodies. For example, cardiac troponin-reactive autoantibodies may interfere with the measurement of cTnI using conventional midfragment-specific immunoassays. Thus, interference from autoantibodies can produce erroneous results, particularly near the cut-off values established for clinical diagnoses, and increases the risk of false negative diagnostic results and the risk that individuals will not obtain a timely diagnosis.

One approach to addressing this problem is to choose analyte-specific antibodies that bind to specific epitopes distinct from the analyte epitopes that react with the autoantibodies. Following this general approach, efforts have focused on exploring the use of thousands of different combinations of two, three and even four analyte-specific antibodies to avoid interference from autoantibodies. However, this effort has been largely unsuccessful. It is now evident that autoantibodies against complex protein analytes are likely to be polyclonal within a particular sample, and may be even more diverse among samples from different individuals. Interference from diverse polyclonal autoantibodies may explain the observation that as little as 25% or even less of an analyte protein sequence binds to analyte-specific antibodies, which may in turn explain the lack of success using this approach.

A need exists in the art for new immunoassay methods that compensate for interference by autoantibodies in a sample, and in particular for such methods that do so without involving redesign of the analyte detection or capture antibodies.

### SUMMARY

In one embodiment, the present disclosure relates to a reagent for use in an immunoassay for determining the presence or amount of at least one protein in a test sample, the reagent comprising at least one peptide comprising at least 5 consecutive amino acid residues wherein the peptide is derived from the protein and further wherein the reagent is used to block the interaction between an endogenous antibody and the protein in the test sample.

In certain embodiments, the protein from which the reagent is derived may be selected from the group consisting of: cardiac troponin I (SEQ ID NO:1), cardiac troponin T (SEQ ID NO:2), thyroid stimulating hormone (TSH) (SEQ ID NO:3), beta-human chorionic gonadotropin (beta-HCG) (SEQ ID NO:4), myeloperoxidase (MPO) (SEQ ID NO:5), prostate specific antigen (PSA) (SEQ ID NO:6), human B-type natriuretic peptide (hBNP) (SEQ ID NO:7), myosin light chain 2 (SEQ ID NO:8), myosin-6 (SEQ ID NO:9) and myosin-7 (SEQ ID NO:10).

The peptide can have, for example, an amino acid sequence of five (5) consecutive amino acid residues to fifteen (15) consecutive amino acid residues from the amino acid sequence of the protein from which the reagent is derived. In one embodiment, for example, the protein from which the reagent is derived is cardiac troponin I, and the reagent has an amino acid sequence comprising at least five consecutive amino acid residues from the full amino acid sequence of cardiac troponin I (SEQ ID NO: 1). In certain embodiments, the peptide reagent has a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15), or any subsequence thereof consisting of at least 5 consecutive amino acid residues.

In another embodiment, the present disclosure relates to a reagent for use in an immunoassay for determining the presence or amount of a cardiac troponin I in a test sample, the reagent comprising a peptide having a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

In another embodiment, the present disclosure relates to a method of detecting at least one protein of interest in a test sample, the method comprising the steps of:

a. preparing a first mixture comprising a test sample suspected of containing at least one protein of interest and at least one reagent, wherein said reagent (1) is at least one peptide comprising at least 5 consecutive amino acid residues derived from said protein that binds to the antibody of interest; and (2) disrupts the interaction between an endogenous antibody in the test sample and the antigen;

b. preparing a second mixture comprising the first mixture and a first specific binding partner, wherein the first specific binding partner comprises an antibody, wherein the antibody binds with the protein of interest to form a first specific binding partner-protein complex; and

c. contacting the second mixture with a second specific binding partner, wherein the second specific binding partner comprises an antibody that has been conjugated to a detectable label and further wherein the second specific binding partner binds to the first specific binding partner-protein complex to form a first specific binding partner-protein-second specific binding partner complex; and

d. measuring the signal generated by or emitted from the detectable label and detecting the protein of interest in the test sample.

In the above-described method, the protein can be selected for example from the group consisting of: cardiac troponin I, cardiac troponin T, thyroid stimulating hormone (TSH), beta-human chorionic gonadotropin (beta-HCG), myeloperoxidase (MPO), prostate specific antigen (PSA), human B-type natriuretic peptide (hBNP), myosin light chain 2, myosin-6 and myosin

In the above-described method the test sample can be whole blood, serum or plasma.

In one embodiment of the method, the first specific binding partner can be immobilized to a solid phase either before or after the formation of the first specific binding partner-protein complex. Additionally, the second specific binding partner can be immobilized to a solid phase either before or after formation of the first specific binding partner-protein-second specific binding partner complex.

In the above-described method the detectable label can be selected from the group consisting of a radioactive label, an enzymatic label, a chemiluminescent label, a fluorescence label, a thermometric label, and an immuno-polymerase chain reaction label.

In one embodiment of the method the detectable label is an acridinium compound. When an acridinium compound is used, the method may further include:

a. generating or providing a source of hydrogen peroxide to the second mixture contacted with a second specific binding partner;

b. adding a basic solution to the mixture of step (a); and

c. measuring the light signal generated or emitted in step (b) and detecting the protein of interest in the sample.

Any acridinium compound can be used in the above-described method. For example, the acridinium compound can be an acridinium-9-carboxamide having a structure according to formula I:

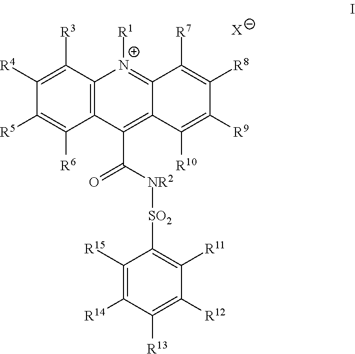

wherein R1 and R2 are each independently selected from the group consisting of: alkyl, alkenyl, alkynyl, aryl or aralkyl, sulfoalkyl, carboxyalkyl and oxoalkyl, and
wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and optionally, if present, XΘ is an anion.

Alternatively, the acridinium compound can be an acridinium-9-carboxylate aryl ester having a structure according to formula II:

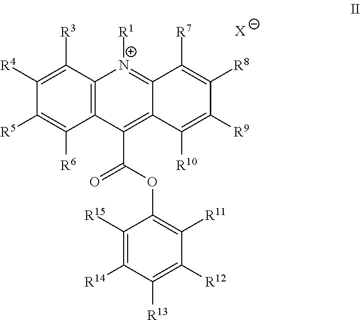

wherein R1 is an alkyl, alkenyl, alkynyl, aryl or aralkyl, sulfoalkyl, carboxyalkyl and oxoalkyl; and

wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, XΘ is an anion.

In the above-described method, the reagent can be a peptide having a length of 5 consecutive amino acids to 15 consecutive amino acids.

In one embodiment of the method, the protein from which the peptide is derived is cardiac troponin I, and the peptide has a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

The above-described method may further include the step of quantifying the amount of protein of interest in the test sample by relating the amount of signal in step (c) to the amount of the one or more proteins of interest in the test sample either by use of a standard curve for the protein of interest or by comparison to a reference standard.

The above-described method may be adapted for use in an automated system or semi-automated system.

In still another embodiment, the present disclosure relates to a kit for detecting and/or quantifying at least one protein of interest in a test sample, the kit comprising the above-described peptide reagent, a capture reagent comprising an antibody that binds to the protein of interest, and instructions for detecting and/or quantifying at least one protein of interest in a test sample.

The above-described kit may further include a conjugate comprising an antibody conjugated to a detectable label.

In one embodiment of the kit, the detectable label can be selected from the group consisting of a radioactive label, an enzymatic label, a chemiluminescent label, a fluorescence label, a thermometric label, and an immuno-polymerase chain reaction label.

The detectable label used in the above-described kit can be an acridinium compound. Any acridinium compound can be used. For example the acridinium compound can be an acridinium-9-carboxamide having a structure according to formula I:

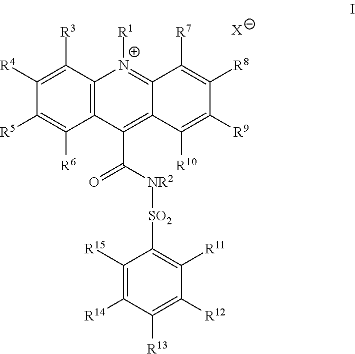

wherein R1 and R2 are each independently selected from the group consisting of: alkyl, alkenyl, alkynyl, aryl or aralkyl, sulfoalkyl, carboxyalkyl and oxoalkyl, and

wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Alternatively, the acridinium compound can be an acridinium-9-carboxylate aryl ester having a structure according to formula II:

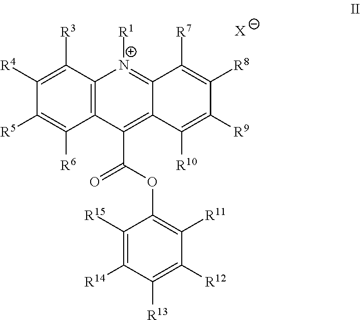

wherein R1 is an alkyl, alkenyl, alkynyl, aryl or aralkyl, sulfoalkyl, carboxyalkyl and oxoalkyl; and

wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, XΘ is an anion.

When an acridinium compound is included as the detectable label in the above-described kit, the kit optionally further includes a basic solution. The basic solution can be for example a solution having a pH of at least about 10.

The above kit may further include a hydrogen peroxide source, which can be a buffer, a solution containing hydrogen peroxide, or a hydrogen peroxide generating enzyme. In kits containing a hydrogen peroxide generating enzyme, the enzyme can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N6-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

In one embodiment, the above-described kit includes a reagent derived from a protein selected from the group consisting of: cardiac troponin I, cardiac troponin T, thyroid stimulating hormone (TSH), beta-human chorionic gonadotropin (beta-HCG), myeloperoxidase (MPO), prostate specific antigen (PSA), human B-type natriuretic peptide (hBNP), myosin light chain 2, myosin-6 and myosin-7.

In the above-described kit, the reagent can be a peptide having a length of 5 consecutive amino acids to 15 consecutive amino acids.

In one embodiment of the above-described kit, the protein from which the reagent is derived is cardiac troponin I, and the peptide has a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

### BRIEF DESCRIPTION OF THE FIGURES

FIG. 1 shows the amino acid sequence of cardiac troponin I;

FIG. 2 shows the amino acid sequence of cardiac troponin T;

FIG. 3 shows the amino acid sequence of thyroid stimulating hormone (TSH);

FIG. 4 shows the amino acid sequence of the beta subunit of human chorionic gonadotropin (beta-HCG);

FIG. 5 shows the amino acid sequence of myeloperoxidase (MPO);

FIG. 6 shows the amino acid sequence of prostate specific antigen (PSA);

FIG. 7 shows the amino acid sequence of human B-type natriuretic peptide (hBNP);

FIG. 8 shows the amino acid sequence of myosin light chain 2;

FIG. 9 A-C shows the amino acid sequence of myosin-6;

FIG. 10 A-C shows the amino acid sequence of myosin-7;

FIG. 11 shows a graph of the ratio of the signal to the low control (S/LC) against concentration (nmol/mL) for each of five different peptide reagents and a combination thereof; and

FIG. 12 shows a graph of the ratio of the signal to the low control (S/LC) against concentration (nmol/mL) for each of five different peptide reagents and a combination thereof.

### DETAILED DESCRIPTION

The present disclosure relates to immunoassay methods and kits for detecting a protein of interest in a test sample, and more particularly to methods and kits for detecting a protein in a human test sample that may contain endogenous antibodies against the protein of interest. Specifically, the inventors have discovered an alternative approach to address the problem of interference by autoantibodies in immunodetection of clinically significant analytes in a sample. Such analytes include self-antigens such as for example cardiac troponin, myeloperoxidase, prostate specific antigen and thyroid stimulating hormone. More specifically, the alternative approach includes use of a peptide reagent that is derived from the protein, especially a self-antigen, of interest. The peptide reagent inhibits binding of autoantibodies to the protein, and thus prevents interference by autoantibodies with immunodetection of the protein. This approach compensates for the presence of autoantibodies that may be in the sample without need for a redesign of the specific detection antibodies or the capture antibodies, does not require use of an extra anti-human IgG detection conjugate, and avoids the need of a second assay to identify problematic samples.

### A. Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Acyl (and Other Chemical Structural Group Definitions)

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present disclosure can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂.

As used herein, the term “carboxyalkyl” refers to a —(CH₂)ₙCO₂H or —(CH₂)ₙCO₂⁻ group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present disclosure can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

b) Anion

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

c) Antibody

As used herein, the term “antibody” refers to a protein consisting of one or more polypeptides substantially encoded by immunoglobulin genes or fragments of immunoglobulin genes, and encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

d) Hydrogen Peroxide Generating Enzyme

As used herein, the term “hydrogen peroxide generating enzyme” refers to an enzyme that is capable of producing as a reaction product the chemical compound having the molecular formula H₂O₂, i.e. hydrogen peroxide. Non-limiting examples of hydrogen peroxide generating enzymes are listed below in Table 1.

TABLE 1

| | IUBMB ENZYME | PREFERRED |
|---|---|---|
| ACCEPTED COMMON NAME | NOMENCLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| Aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol |
| | | (2-naphthyl)methanol |
| | | 3-methoxybenzyl |
| | | alcohol |
| aryl-aldehyde oxidase | EC 1.2.3.9 | an aromatic aldehyde |
| Catechol oxidase | EC 1.1.3.14 | Catechol |
| cholesterol oxidase | EC 1.1.3.6 | Cholesterol |
| Choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| Ecdysone oxidase | EC 1.1.3.16 | Ecdysone |
| ethanolamine oxidase | EC 1.4.3.8 | Ethanolamine |
| Galactose oxidase | EC 1.1.3.9 | D-galactose |
| Glucose oxidase | EC 1.1.3.4 | β-D-glucose |
| glutathione oxidase | EC 1.8.3.3 | Glutathione |
| Glycerol-3-phosphate oxidase | EC 1.1.3.21 | sn-glycerol 3-phosphate |
| Glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| Oxalate oxidase | EC 1.2.3.4 | Oxalate |
| polyamine oxidase | EC 1.5.3.11 | 1-N-acetylspermine |
| polyphenol oxidase | EC 1.14.18.1 | |
| Polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| Pyranose oxidase | EC 1.1.3.10 | D-glucose |
| | | D-xylose |
| | | L-sorbose |
| | | D-glucono-1,5-lactone |
| Pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8- |
| | | dioxo-1,2,3,4,5,6,7,8- |
| | | octahydroquinoline-2,4- |
| | | dicarboxylate |
| Pyruvate oxidase | EC 1.2.3.3 | Pyruvate |
| Pyruvate oxidase (CoA-acetylating) | EC 1.2.3.6 | Pyruvate |
| Reticuline oxidase | EC 1.21.3.3 | Reticuline |
| retinal oxidase | EC 1.2.3.11 | Retinal |
| Rifamycin-B oxidase | EC 1.10.3.6 | rifamycin-B |
| Sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| Thiamine oxidase | EC 1.1.3.23 | Thiamine |
| tryptophan α,β-oxidase | EC 1.3.3.10 | L-tryptophan |
| urate oxidase (uricase, uric acid oxidase) | EC 1.7.3.3 | uric acid |
| Vanillyl-alcohol oxidase | EC 1.1.3.38 | vanillyl alcohol |
| Xanthine oxidase | EC 1.17.3.2 | Xanthine |
| xylitol oxidase | EC 1.1.3.41 | Xylitol |

e) Autoantibody

As used herein, the phrase “autoantibody” refers to an antibody that binds to an analyte that is endogenously produced in the subject in which the antibody is produced.

f) Specific Binding Partner

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

g) Specific Binding Partner-Protein Complex

As used herein, the phrase “specific binding partner-protein complex” refers to a combination of an antibody and an antigen, in which the antigen is a protein of interest, and the antibody and protein are bound by specific, noncovalent interactions between an antigen-combining site on the antibody and an antigen epitope.

h) Detectable Label

As used herein the term “detectable label” refers to any moiety that generates a measurable signal via optical, electrical, or other physical indication of a change of state of a molecule or molecules coupled to the moiety. Such physical indicators encompass spectroscopic, photochemical, biochemical, immunochemical, electromagnetic, radiochemical, and chemical means, such as but not limited to fluorescence, chemifluorescence, chemiluminescence, and the like. Preferred detectable labels include acridinium compounds such as an acridinium-9-carboximide having a structure according to Formula I as set forth in section B herein below, and an acridinium-9-carboxylate aryl ester having a structure according to Formula II as also set forth in section B herein below.

i) Subject

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the subject is a human.

j) Test Sample

As used herein, the term “test sample” generally refers to a biological material being tested for and/or suspected of containing an protein of interest and which may also include autoantibodies to the protein of interest. The biological material may be derived from any biological source but preferably is a biological fluid likely to contain the protein of interest. Examples of biological materials include, but are not limited to, stool, whole blood, serum, plasma, red blood cells, platelets, interstitial fluid, saliva, ocular lens fluid, cerebral spinal fluid, sweat, urine, ascites fluid, mucous, nasal fluid, sputum, synovial fluid, peritoneal fluid, vaginal fluid, menses, amniotic fluid, semen, soil, etc. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. If such methods of pretreatment are employed with respect to the test sample, such pretreatment methods are such that the protein of interest remains in the test sample at a concentration proportional to that in an untreated test sample (e.g., namely, a test sample that is not subjected to any such pretreatment method(s)).

### B. Peptide Reagents

Self-antigens include a number of proteins that are known to be endogenously produced in relation to a particular disease state or injury in a subject. Self-antigens for which autoantibodies have been identified include the troponins, namely cardiac troponin I (SEQ ID NO:1), and cardiac troponin T (SEQ ID NO:2); thyroid stimulating hormone (TSH) (SEQ ID NO:3); the beta subunit of human chorionic gonadotropin (beta-HCG) (SEQ ID NO:4); myeloperoxidase (MPO) (SEQ ID NO:5); prostate specific antigen (PSA) (SEQ ID NO:6); human B-type natriuretic peptide (hBNP) (SEQ ID NO:7); myosin light chain 2 (SEQ ID NO:8); myosin-6 (SEQ ID NO:9) and myosin-7 (SEQ ID NO:10).

The peptide reagents of the present disclosure are derived from the amino acid sequence of the target self-antigen, and can be used in an immunoassay format to prevent interference by autoantibodies against the self-antigen. More specifically, the peptide reagent is used to block the interaction between the self-antigen and any autoantibodies against the self-antigen that may be present in a test sample. Each peptide reagent may be used alone, or in combination with one or more other peptide reagents derived from the target protein. A synergistic blocking effect is believed to result from a combination of different peptide reagents derived from the same target protein.

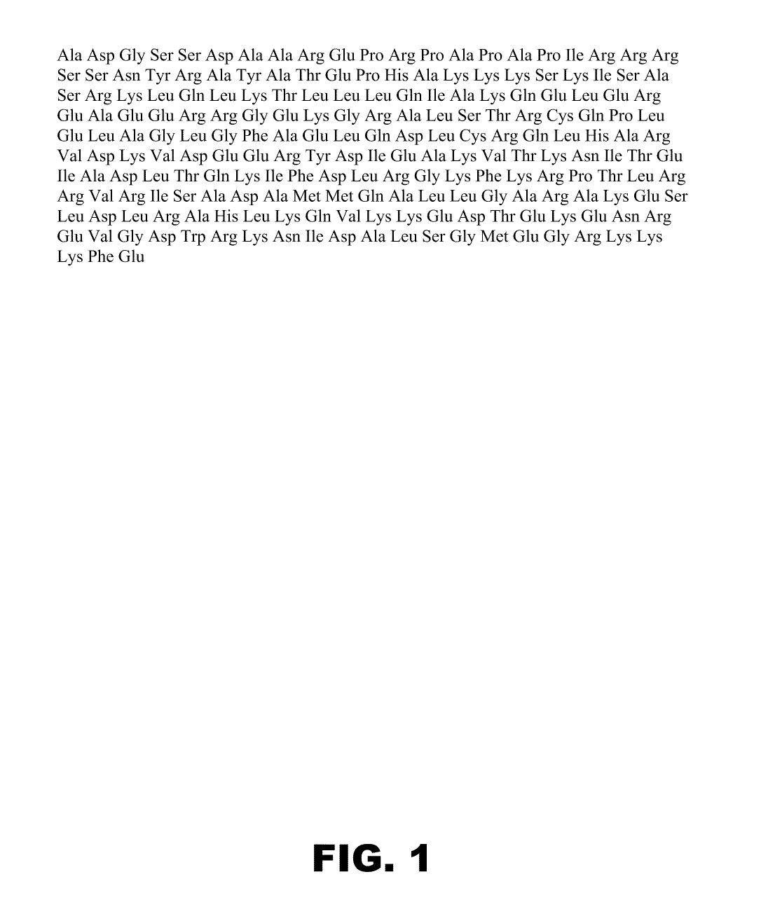

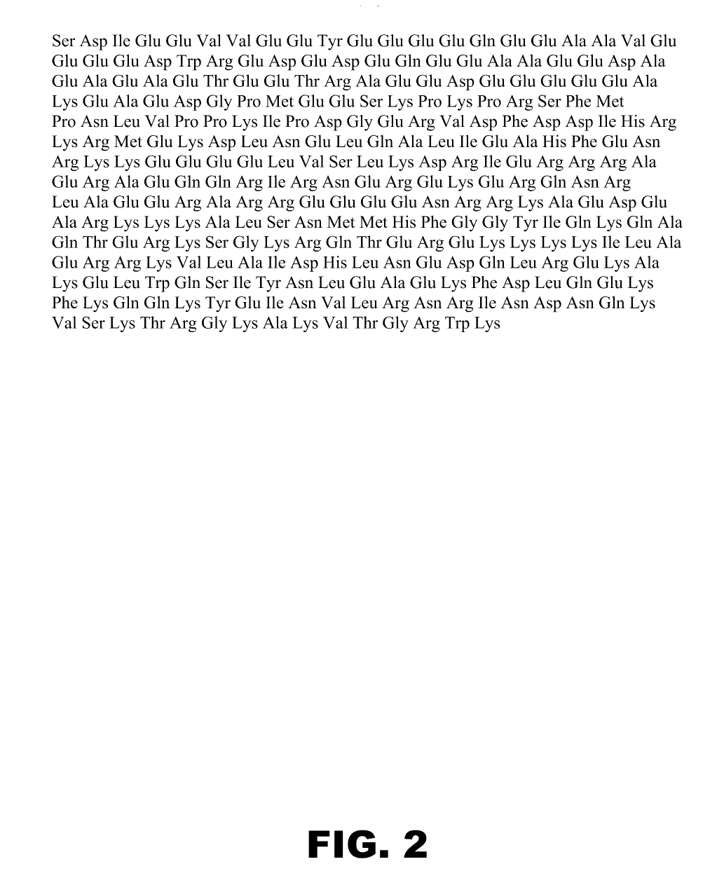

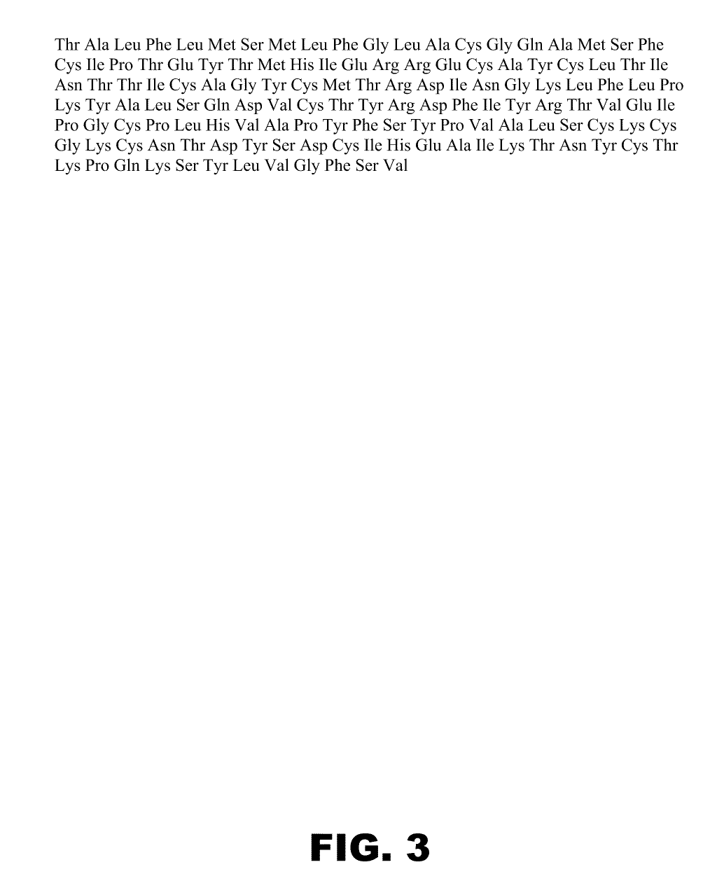

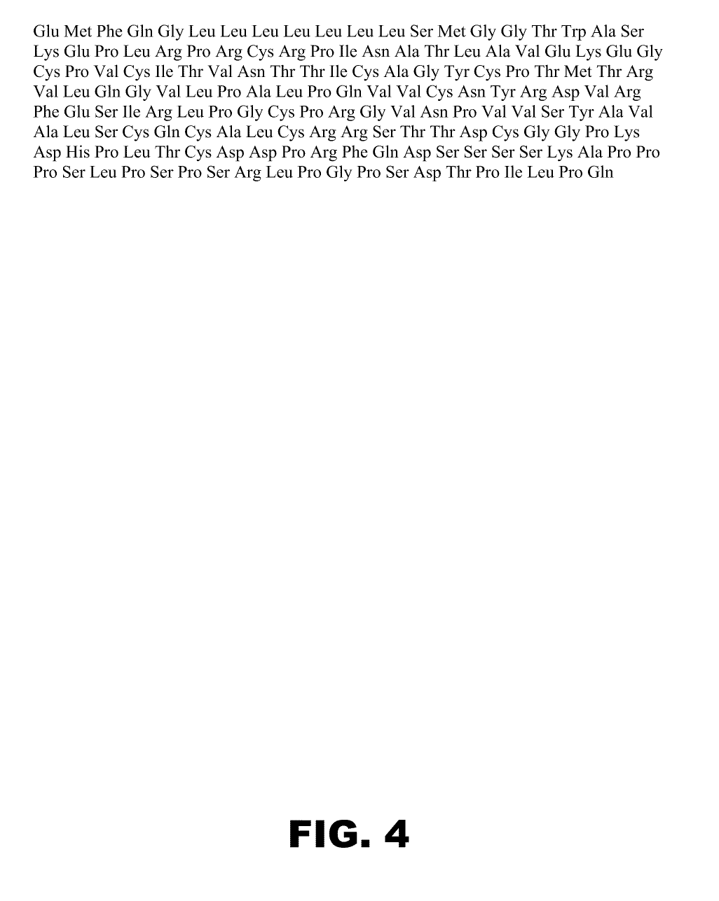

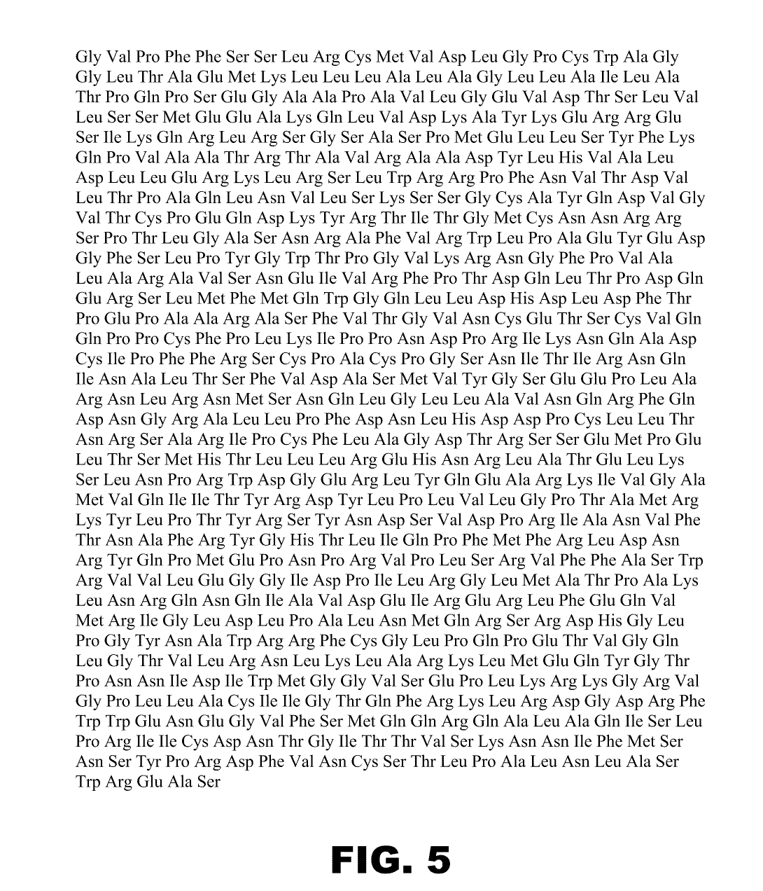

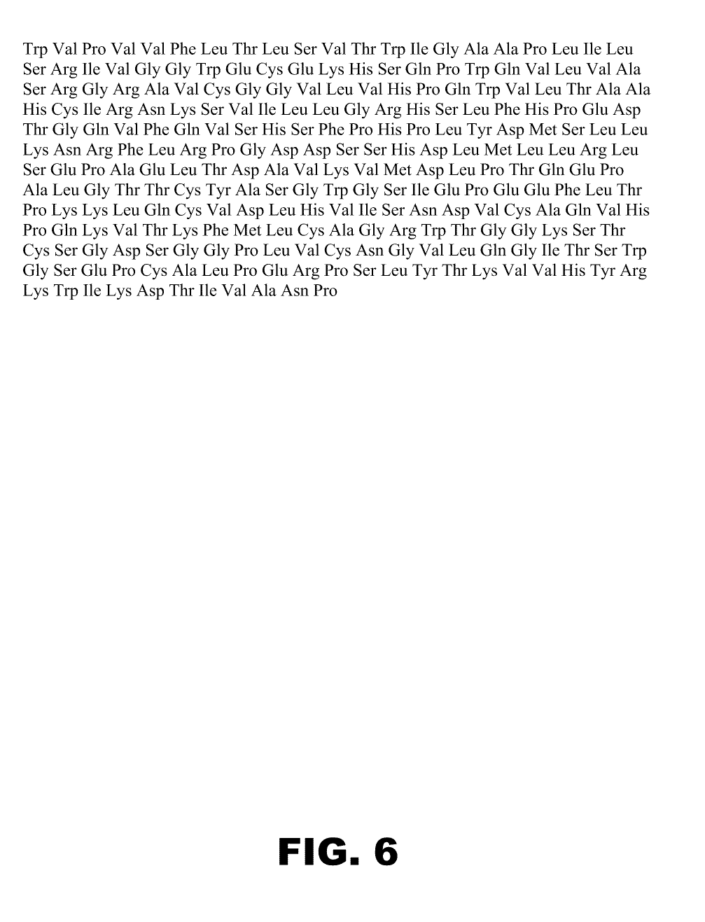

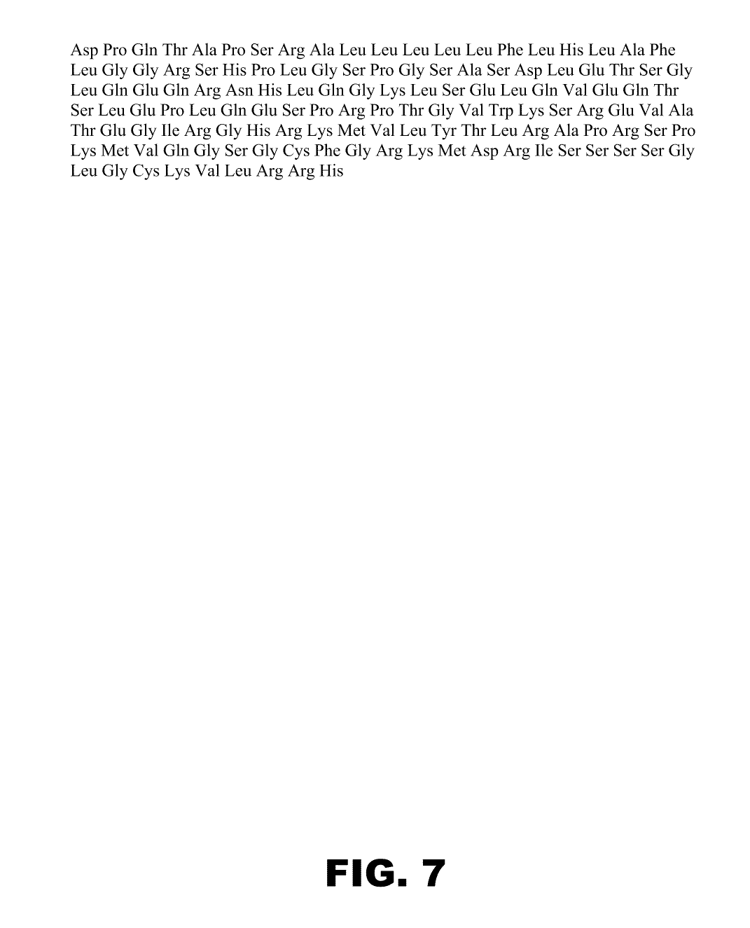

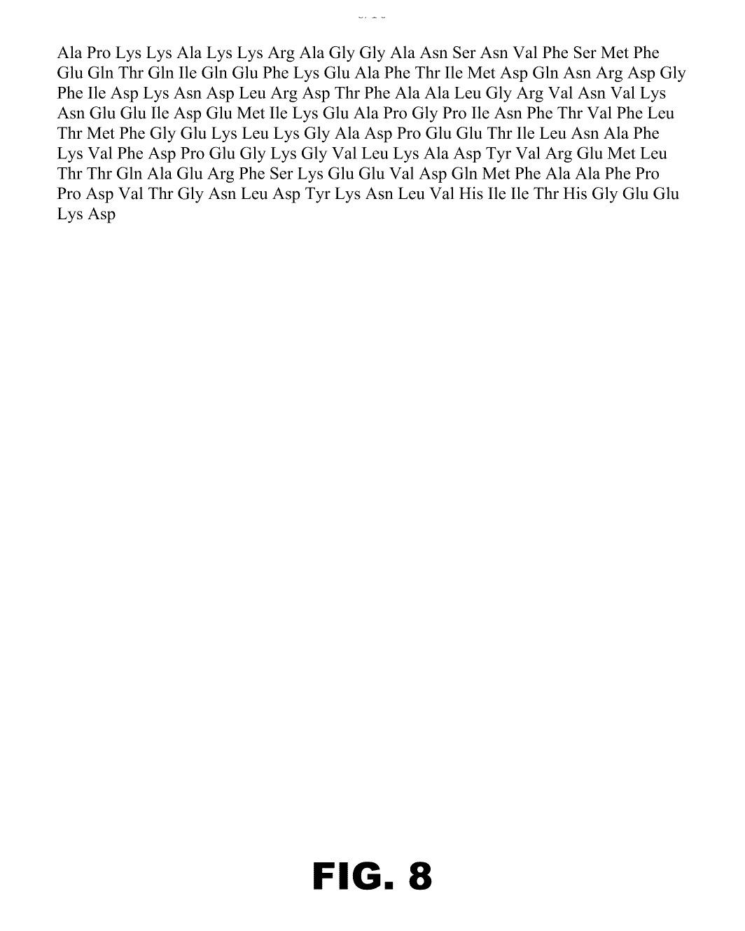

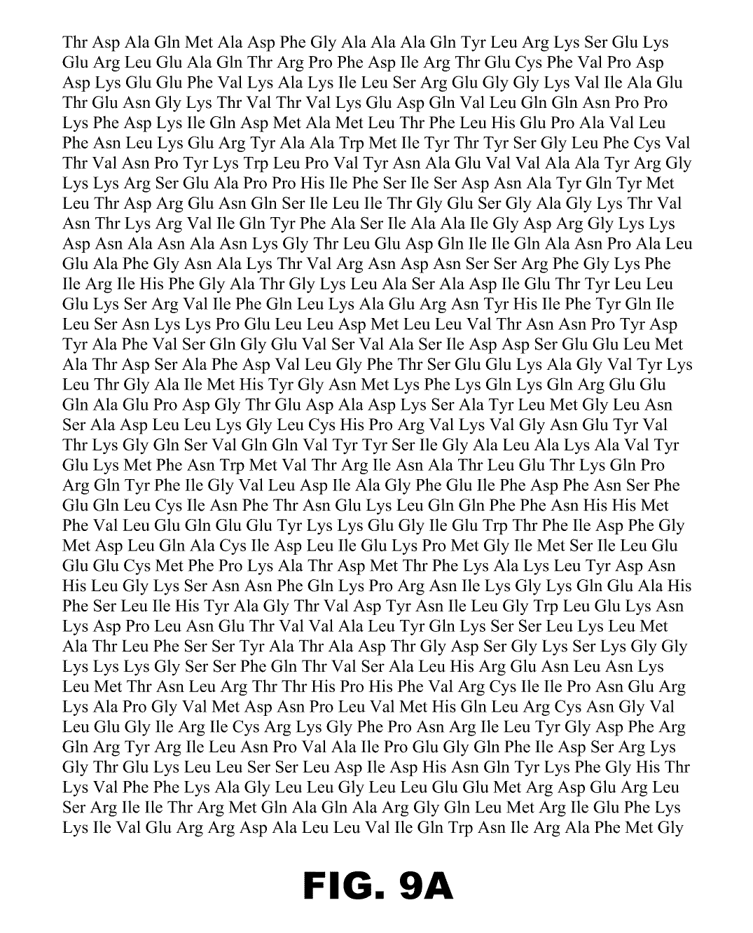

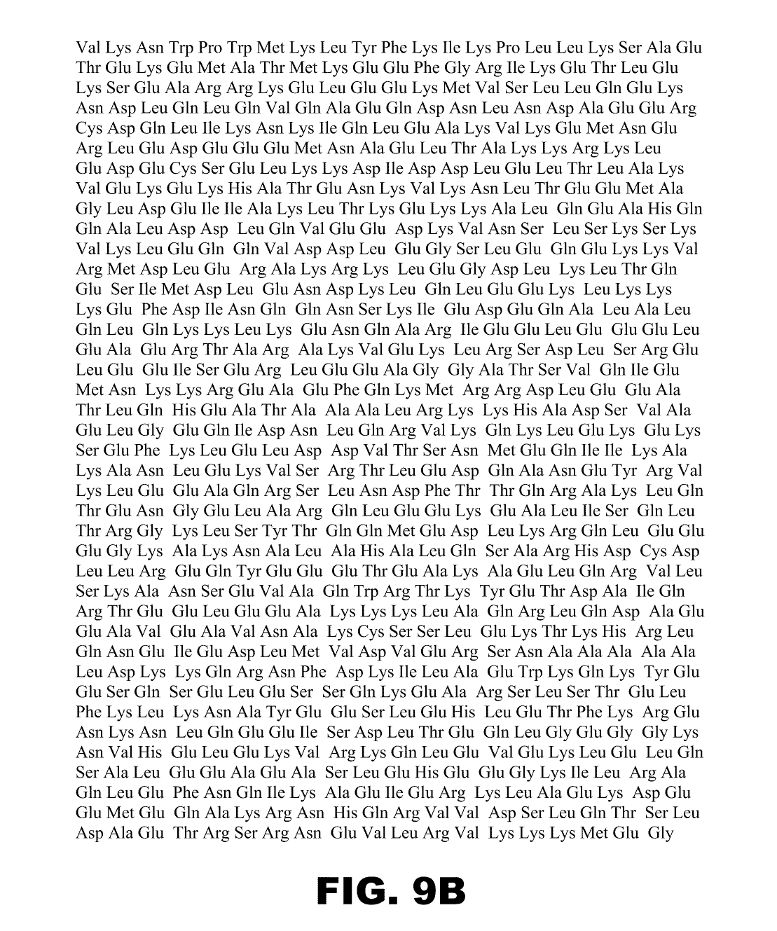

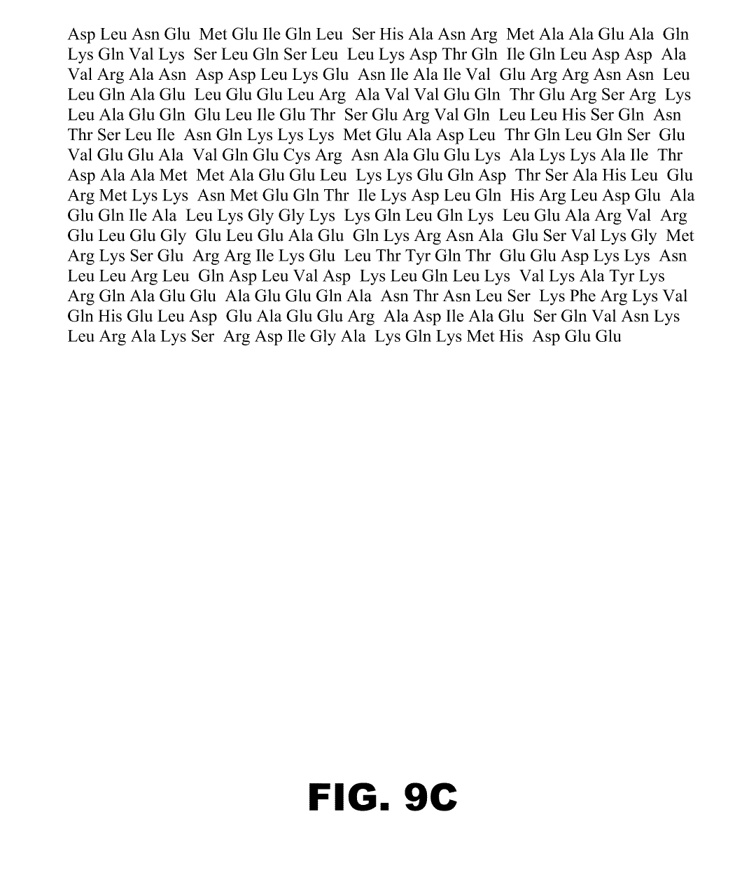

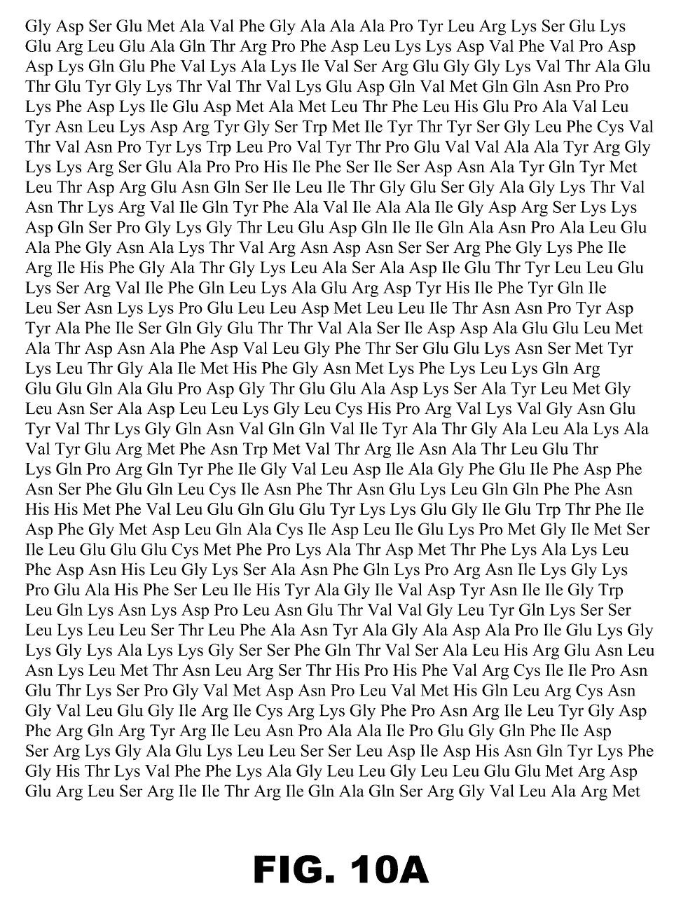

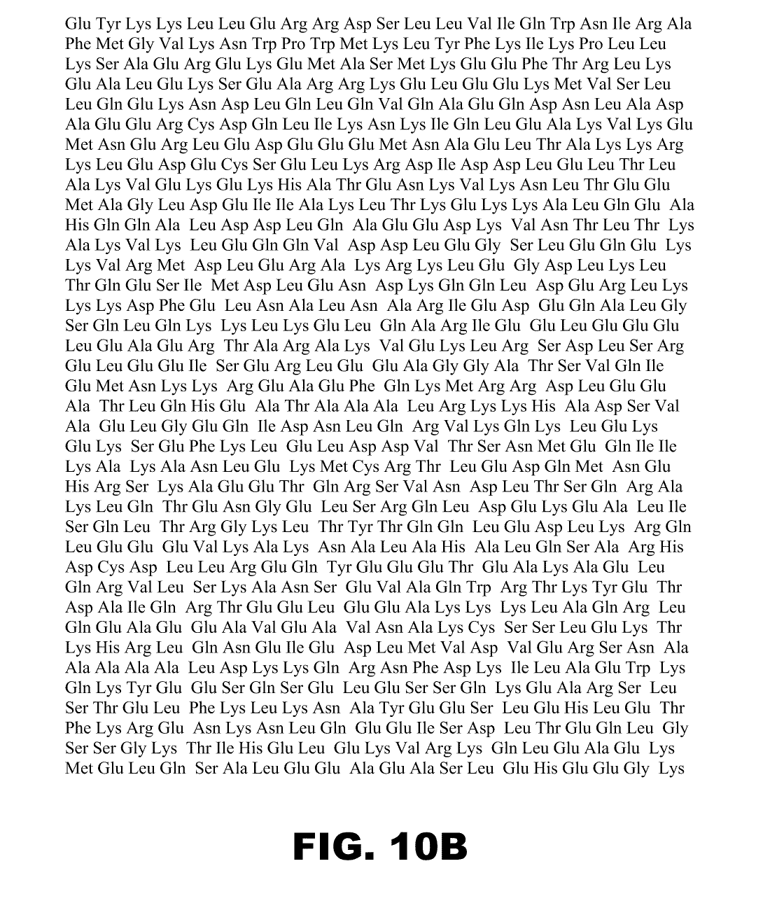

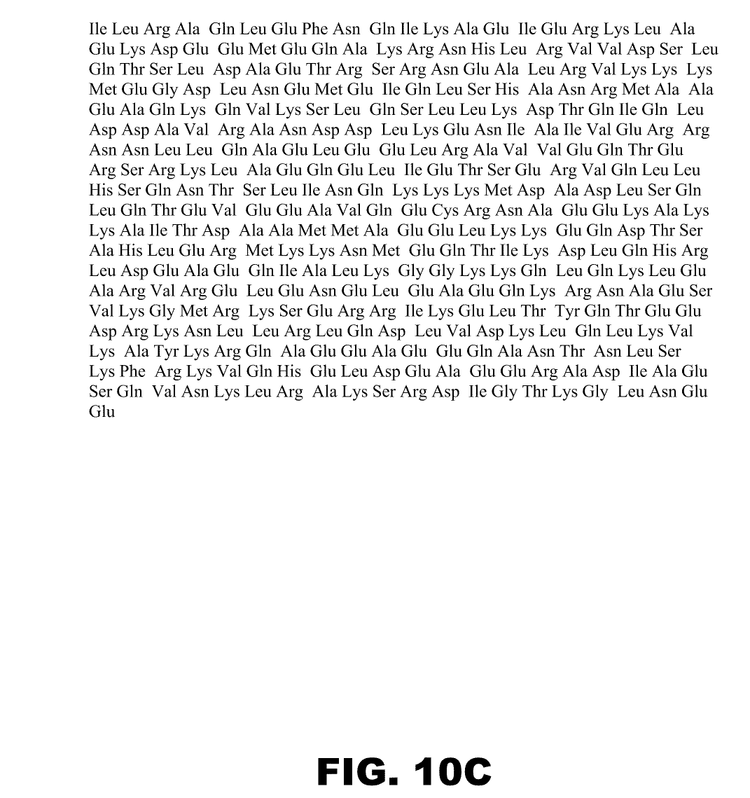

The peptide reagent includes at least five (5) consecutive amino acid residues from the amino acid sequence of the target self-antigen. In one embodiment, the peptide reagent includes five (5) to fifteen (15) consecutive amino acid residues from the amino acid sequence of the target self-antigen. For example, given cardiac troponin I as the target self-antigen, the peptide reagent comprises any sequence of 5 to 15 consecutive amino acid residues from anywhere in the amino acid sequence of cardiac troponin I (FIG. 1; SEQ ID NO: 1). For example, the peptide reagent can comprise any of the following amino acid sequences: ADGSS (residues 1-5), KFFES (residues 205-209), or KKKSKISASRKLQLK (residues 35-49), or any other sequence of 5 to 15 consecutive amino acid residues from anywhere in the amino acid sequence of cardiac troponin I (SEQ ID NO: 1). Table 2 lists amino acid sequences for exemplary peptide reagents consisting of 5 consecutive amino acid residues from cardiac troponin I (SEQ ID NO: 1). Additional peptide reagents may have a length of up to 15 amino acid residues, comprising any one of the listed 5-amino acid long sequences in Table 2, plus up to a total of 10 additional consecutive amino acid residues from SEQ ID NO:1, that are continuous (from either side within the protein amino sequence) with the 5-amino acid long sequence.

TABLE 2

| | | |
|---|---|---|
| Ala Asp Gly Ser Ser | Leu Leu Leu Gln Ile | Ala Arg Val Asp Lys |
| Asp Gly Ser Ser Asp | Leu Leu Gln Ile Ala | Arg Val Asp Lys Val |
| Gly Ser Ser Asp Ala | Leu Gln Ile Ala Lys | Val Asp Lys Val Asp |
| Ser Ser Asp Ala Ala | Gln Ile Ala Lys Gln | Asp Lys Val Asp Glu |
| Ser Asp Ala Ala Arg | Ile Ala Lys Gln Glu | Lys Val Asp Glu Glu |
| Asp Ala Ala Arg Glu | Ala Lys Gln Glu Leu | Val Asp Glu Glu Arg |
| Ala Ala Arg Glu Pro | Lys Gln Glu Leu Glu | Asp Glu Glu Arg Tyr |
| Ala Arg Glu Pro Arg | Gln Glu Leu Glu Arg | Glu Glu Arg Tyr Asp |
| Arg Glu Pro Arg Pro | Glu Leu Glu Arg Glu | Glu Arg Tyr Asp Ile |
| Glu Pro Arg Pro Ala | Leu Glu Arg Glu Ala | Arg Tyr Asp Ile Glu |
| Pro Arg Pro Ala Pro | Glu Arg Glu Ala Glu | Tyr Asp Ile Glu Ala |
| Arg Pro Ala Pro Ala | Arg Glu Ala Glu Glu | Asp Ile Glu Ala Lys |
| Pro Ala Pro Ala Pro | Glu Ala Glu Glu Arg | Ile Glu Ala Lys Val |
| Ala Pro Ala Pro Ile | Ala Glu Glu Arg Arg | Glu Ala Lys Val Thr |
| Pro Ala Pro Ile Arg | Glu Glu Arg Arg Gly | Ala Lys Val Thr Lys |
| Ala Pro Ile Arg Arg | Glu Arg Arg Gly Glu | Lys Val Thr Lys Asn |
| Pro Ile Arg Arg Arg | Arg Arg Gly Glu Lys | Val Thr Lys Asn Ile |
| Ile Arg Arg Arg Ser | Arg Gly Glu Lys Gly | Thr Lys Asn Ile Thr |
| Arg Arg Arg Ser Ser | Gly Glu Lys Gly Arg | Lys Asn Ile Thr Glu |
| Arg Arg Ser Ser Asn | Glu Lys Gly Arg Ala | Asn Ile Thr Glu Ile |
| Arg Ser Ser Asn Tyr | Lys Gly Arg Ala Leu | Ile Thr Glu Ile Ala |
| Ser Ser Asn Tyr Arg | Gly Arg Ala Leu Ser | Thr Glu Ile Ala Asp |
| Ser Asn Tyr Arg Ala | Arg Ala Leu Ser Thr | Glu Ile Ala Asp Leu |
| Asn Tyr Arg Ala Tyr | Ala Leu Ser Thr Arg | Ile Ala Asp Leu Thr |
| Tyr Arg Ala Tyr Ala | Leu Ser Thr Arg Cys | Ala Asp Leu Thr Gln |
| Arg Ala Tyr Ala Thr | Ser Thr Arg Cys Gln | Asp Leu Thr Gln Lys |
| Ala Tyr Ala Thr Glu | Thr Arg Cys Gln Pro | Leu Thr Gln Lys Ile |
| Tyr Ala Thr Glu Pro | Arg Cys Gln Pro Leu | Thr Gln Lys Ile Phe |
| Ala Thr Glu Pro His | Cys Gln Pro Leu Glu | Gln Lys Ile Phe Asp |
| Thr Glu Pro His Ala | Gln Pro Leu Glu Leu | Lys Ile Phe Asp Leu |
| Glu Pro His Ala Lys | Pro Leu Glu Leu Ala | Ile Phe Asp Leu Arg |
| Pro His Ala Lys Lys | Leu Glu Leu Ala Gly | Phe Asp Leu Arg Gly |
| His Ala Lys Lys Lys | Glu Leu Ala Gly Leu | Asp Leu Arg Gly Lys |
| Ala Lys Lys Lys Ser | Leu Ala Gly Leu Gly | Leu Arg Gly Lys Phe |
| Lys Lys Lys Ser Lys | Ala Gly Leu Gly Phe | Arg Gly Lys Phe Lys |
| Lys Lys Ser Lys Ile | Gly Leu Gly Phe Ala | Gly Lys Phe Lys Arg |
| Lys Ser Lys Ile Ser | Leu Gly Phe Ala Glu | Lys Phe Lys Arg Pro |
| Ser Lys Ile Ser Ala | Gly Phe Ala Glu Leu | Phe Lys Arg Pro Thr |
| Lys Ile Ser Ala Ser | Phe Ala Glu Leu Gln | Lys Arg Pro Thr Leu |
| Ile Ser Ala Ser Arg | Ala Glu Leu Gln Asp | Arg Pro Thr Leu Arg |
| Ser Ala Ser Arg Lys | Glu Leu Gln Asp Leu | |
| Ala Ser Arg Lys Leu | Leu Gln Asp Leu Cys | |
| Ser Arg Lys Leu Gln | Gln Asp Leu Cys Arg | |
| Arg Lys Leu Gln Leu | Asp Leu Cys Arg Gln | |
| Lys Leu Gln Leu Lys | Leu Cys Arg Gln Leu | |
| Leu Gln Leu Lys Thr | Cys Arg Gln Leu His | |
| Gln Leu Lys Thr Leu | Arg Gln Leu His Ala | |
| Leu Lys Thr Leu Leu | Gln Leu His Ala Arg | |
| Lys Thr Leu Leu Leu | Leu His Ala Arg Val | |
| Thr Leu Leu Leu Gln | His Ala Arg Val Asp | |

When cardiac troponin T (SEQ ID NO:2) is the target self-antigen, the peptide reagent comprises any sequence of 5 to 15 consecutive amino acid residues from anywhere in the amino acid sequence of cardiac troponin T (SEQ ID NO: 2). Table 3 lists amino acid sequences for exemplary peptide reagents consisting of 5 consecutive amino acid residues from cardiac troponin T (SEQ ID NO: 2). Additional peptide reagents may have a length of up to 15 amino acid residues, comprising any one of the listed 5-amino acid long sequences in Table 3, plus up to a total of 10 additional consecutive amino acid residues from SEQ ID NO:2, that are continuous (from either side within the protein amino sequence) with the 5-amino acid long sequence.

TABLE 3

| | |
|---|---|
| Ser Asp Ile Glu Glu | Arg Ala Glu Glu Asp |
| Asp Ile Glu Glu Val | Ala Glu Glu Asp Glu |
| Ile Glu Glu Val Val | Glu Glu Asp Glu Glu |
| Glu Glu Val Val Glu | Glu Asp Glu Glu Glu |
| Glu Val Val Glu Glu | Asp Glu Glu Glu Glu |
| Val Val Glu Glu Tyr | Glu Glu Glu Glu Glu |
| Val Glu Glu Tyr Glu | Glu Glu Glu Glu Ala |
| Glu Glu Tyr Glu Glu | Glu Glu Glu Ala Lys |
| Glu Tyr Glu Glu Glu | Glu Glu Ala Lys Glu |
| Tyr Glu Glu Glu Glu | Glu Ala Lys Glu Ala |
| Glu Glu Glu Glu Gln | Ala Lys Glu Ala Glu |
| Glu Glu Glu Gln Glu | Lys Glu Ala Glu Asp |
| Glu Glu Gln Glu Glu | Glu Ala Glu Asp Gly |
| Glu Gln Glu Glu Ala | Ala Glu Asp Gly Pro |
| Gln Glu Glu Ala Ala | Glu Asp Gly Pro Met |
| Glu Glu Ala Ala Val | Asp Gly Pro Met Glu |
| Glu Ala Ala Val Glu | Gly Pro Met Glu Glu |
| Ala Ala Val Glu Glu | Pro Met Glu Glu Ser |
| Ala Val Glu Glu Glu | Met Glu Glu Ser Lys |
| Val Glu Glu Glu Glu | Glu Glu Ser Lys Pro |
| Glu Glu Glu Glu Asp | Glu Ser Lys Pro Lys |
| Glu Glu Glu Asp Trp | Ser Lys Pro Lys Pro |
| Glu Glu Asp Trp Arg | Lys Pro Lys Pro Arg |
| Glu Asp Trp Arg Glu | Pro Lys Pro Arg Ser |
| Asp Trp Arg Glu Asp | Lys Pro Arg Ser Phe |
| Trp Arg Glu Asp Glu | Pro Arg Ser Phe Met |
| Arg Glu Asp Glu Asp | |
| Glu Asp Glu Asp Glu | |
| Asp Glu Asp Glu Gln | |
| Glu Asp Glu Gln Glu | |
| Asp Glu Gln Glu Glu | |
| Glu Gln Glu Glu Ala | |
| Gln Glu Glu Ala Ala | |
| Glu Glu Ala Ala Glu | |
| Glu Ala Ala Glu Glu | |
| Ala Ala Glu Glu Asp | |
| Ala Glu Glu Asp Ala | |
| Glu Glu Asp Ala Glu | |
| Glu Asp Ala Glu Ala | |
| Asp Ala Glu Ala Glu | |
| Ala Glu Ala Glu Ala | |
| Glu Ala Glu Ala Glu | |
| Ala Glu Ala Glu Thr | |
| Glu Ala Glu Thr Glu | |
| Ala Glu Thr Glu Glu | |
| Glu Thr Glu Glu Thr | |
| Thr Glu Glu Thr Arg | |
| Glu Glu Thr Arg Ala | |
| Glu Thr Arg Ala Glu | |
| Thr Arg Ala Glu Glu | |

When thyroid stimulating hormone (TSH) (SEQ ID NO:3) is the target self-antigen, the peptide reagent comprises any sequence of 5 to 15 consecutive amino acid residues from anywhere in the amino acid sequence of (TSH) (SEQ ID NO:3). Table 3 lists amino acid sequences for exemplary peptide reagents consisting of 5 consecutive amino acid residues from TSH (SEQ ID NO: 3). Additional peptide reagents may have a length of up to 15 amino acid residues, comprising any one of the listed 5-amino acid long sequences in Table 4, plus up to a total of 10 additional consecutive amino acid residues from SEQ ID NO:3, that are continuous (from either side within the protein amino sequence) with the 5-amino acid long sequence.

TABLE 4

| | | |
|---|---|---|
| Thr Ala Leu Phe Leu | Met Thr Arg Asp Ile | Ser Cys Lys Cys Gly |
| Ala Leu Phe Leu Met | Thr Arg Asp Ile Asn | Cys Lys Cys Gly Lys |
| Leu Phe Leu Met Ser | Arg Asp Ile Asn Gly | Lys Cys Gly Lys Cys |
| Phe Leu Met Ser Met | Asp Ile Asn Gly Lys | Cys Gly Lys Cys Asn |
| Leu Met Ser Met Leu | Ile Asn Gly Lys Leu | Gly Lys Cys Asn Thr |
| Met Ser Met Leu Phe | Asn Gly Lys Leu Phe | Lys Cys Asn Thr Asp |
| Ser Met Leu Phe Gly | Gly Lys Leu Phe Leu | Cys Asn Thr Asp Tyr |
| Met Leu Phe Gly Leu | Lys Leu Phe Leu Pro | Asn Thr Asp Tyr Ser |
| Leu Phe Gly Leu Ala | Leu Phe Leu Pro Lys | Thr Asp Tyr Ser Asp |
| Phe Gly Leu Ala Cys | Phe Leu Pro Lys Tyr | Asp Tyr Ser Asp Cys |
| Gly Leu Ala Cys Gly | Leu Pro Lys Tyr Ala | Tyr Ser Asp Cys Ile |
| Leu Ala Cys Gly Gln | Pro Lys Tyr Ala Leu | Ser Asp Cys Ile His |
| Ala Cys Gly Gln Ala | Lys Tyr Ala Leu Ser | Asp Cys Ile His Glu |
| Cys Gly Gln Ala Met | Tyr Ala Leu Ser Gln | Cys Ile His Glu Ala |
| Gly Gln Ala Met Ser | Ala Leu Ser Gln Asp | Ile His Glu Ala Ile |
| Gln Ala Met Ser Phe | Leu Ser Gln Asp Val | His Glu Ala Ile Lys |
| Ala Met Ser Phe Cys | Ser Gln Asp Val Cys | Glu Ala Ile Lys Thr |
| Met Ser Phe Cys Ile | Gln Asp Val Cys Thr | Ala Ile Lys Thr Asn |
| Ser Phe Cys Ile Pro | Asp Val Cys Thr Tyr | Ile Lys Thr Asn Tyr |
| Phe Cys Ile Pro Thr | Val Cys Thr Tyr Arg | Lys Thr Asn Tyr Cys |
| Cys Ile Pro Thr Glu | Cys Thr Tyr Arg Asp | Thr Asn Tyr Cys Thr |
| Ile Pro Thr Glu Tyr | Thr Tyr Arg Asp Phe | Asn Tyr Cys Thr Lys |
| Pro Thr Glu Tyr Thr | Tyr Arg Asp Phe Ile | Tyr Cys Thr Lys Pro |
| Thr Glu Tyr Thr Met | Arg Asp Phe Ile Tyr | Cys Thr Lys Pro Gln |
| Glu Tyr Thr Met His | Asp Phe Ile Tyr Arg | Thr Lys Pro Gln Lys |
| Tyr Thr Met His Ile | Phe Ile Tyr Arg Thr | Lys Pro Gln Lys Ser |
| Thr Met His Ile Glu | Ile Tyr Arg Thr Val | Pro Gln Lys Ser Tyr |
| Met His Ile Glu Arg | Tyr Arg Thr Val Glu | Gln Lys Ser Tyr Leu |
| His Ile Glu Arg Arg | Arg Thr Val Glu Ile | Lys Ser Tyr Leu Val |
| Ile Glu Arg Arg Glu | Thr Val Glu Ile Pro | Ser Tyr Leu Val Gly |
| Glu Arg Arg Glu Cys | Val Glu Ile Pro Gly | Tyr Leu Val Gly Phe |
| Arg Arg Glu Cys Ala | Glu Ile Pro Gly Cys | Leu Val Gly Phe Ser |
| Arg Glu Cys Ala Tyr | Ile Pro Gly Cys Pro | Val Gly Phe Ser Val |
| Glu Cys Ala Tyr Cys | Pro Gly Cys Pro Leu | |
| Cys Ala Tyr Cys Leu | Gly Cys Pro Leu His | |
| Ala Tyr Cys Leu Thr | Cys Pro Leu His Val | |
| Tyr Cys Leu Thr Ile | Pro Leu His Val Ala | |
| Cys Leu Thr Ile Asn | Leu His Val Ala Pro | |
| Leu Thr Ile Asn Thr | His Val Ala Pro Tyr | |
| Thr Ile Asn Thr Thr | Val Ala Pro Tyr Phe | |
| Ile Asn Thr Thr Ile | Ala Pro Tyr Phe Ser | |
| Asn Thr Thr Ile Cys | Pro Tyr Phe Ser Tyr | |
| Thr Thr Ile Cys Ala | Tyr Phe Ser Tyr Pro | |
| Thr Ile Cys Ala Gly | Phe Ser Tyr Pro Val | |
| Ile Cys Ala Gly Tyr | Ser Tyr Pro Val Ala | |
| Cys Ala Gly Tyr Cys | Tyr Pro Val Ala Leu | |
| Ala Gly Tyr Cys Met | Pro Val Ala Leu Ser | |
| Gly Tyr Cys Met Thr | Val Ala Leu Ser Cys | |
| Tyr Cys Met Thr Arg | Ala Leu Ser Cys Lys | |
| Cys Met Thr Arg Asp | Leu Ser Cys Lys Cys | |

When the beta subunit of human chorionic gonadotropin (beta-HCG) (SEQ ID NO:4) is the target self-antigen, the peptide reagent comprises any sequence of 5 to 15 consecutive amino acid residues from anywhere in the amino acid sequence of beta-HCG (SEQ ID NO:4). Table 5 lists amino acid sequences for exemplary peptide reagents consisting of 5 consecutive amino acid residues from beta-HCG (SEQ ID NO: 4). Additional peptide reagents may have a length of up to 15 amino acid residues, comprising any one of the listed 5-amino acid long sequences in Table 5, plus up to a total of 10 additional consecutive amino acid residues from SEQ ID NO:4, that are continuous (from either side within the protein amino sequence) with the 5-amino acid long sequence.

TABLE 5

| | | |
|---|---|---|
| Glu Met Phe Gln Gly | Thr Ile Cys Ala Gly | Tyr Ala Val Ala Leu |
| Met Phe Gln Gly Leu | Ile Cys Ala Gly Tyr | Ala Val Ala Leu Ser |
| Phe Gln Gly Leu Leu | Cys Ala Gly Tyr Cys | Val Ala Leu Ser Cys |
| Gln Gly Leu Leu Leu | Ala Gly Tyr Cys Pro | Ala Leu Ser Cys Gln |
| Gly Leu Leu Leu Leu | Gly Tyr Cys Pro Thr | Leu Ser Cys Gln Cys |
| Leu Leu Leu Leu Leu | Tyr Cys Pro Thr Met | Ser Cys Gln Cys Ala |
| Leu Leu Leu Leu Leu | Cys Pro Thr Met Thr | Cys Gln Cys Ala Leu |
| Leu Leu Leu Leu Leu | Pro Thr Met Thr Arg | Gln Cys Ala Leu Cys |
| Leu Leu Leu Leu Ser | Thr Met Thr Arg Val | Cys Ala Leu Cys Arg |
| Leu Leu Leu Ser Met | Met Thr Arg Val Leu | Ala Leu Cys Arg Arg |
| Leu Leu Ser Met Gly | Thr Arg Val Leu Gln | Leu Cys Arg Arg Ser |
| Leu Ser Met Gly Gly | Arg Val Leu Gln Gly | Cys Arg Arg Ser Thr |
| Ser Met Gly Gly Thr | Val Leu Gln Gly Val | Arg Arg Ser Thr Thr |
| Met Gly Gly Thr Trp | Leu Gln Gly Val Leu | Arg Ser Thr Thr Asp |
| Gly Gly Thr Trp Ala | Gln Gly Val Leu Pro | Ser Thr Thr Asp Cys |
| Gly Thr Trp Ala Ser | Gly Val Leu Pro Ala | Thr Thr Asp Cys Gly |
| Thr Trp Ala Ser Lys | Val Leu Pro Ala Leu | Thr Asp Cys Gly Gly |
| Trp Ala Ser Lys Glu | Leu Pro Ala Leu Pro | Asp Cys Gly Gly Pro |
| Ala Ser Lys Glu Pro | Pro Ala Leu Pro Gln | Cys Gly Gly Pro Lys |
| Ser Lys Glu Pro Leu | Ala Leu Pro Gln Val | Gly Gly Pro Lys Asp |
| Lys Glu Pro Leu Arg | Leu Pro Gln Val Val | Gly Pro Lys Asp His |
| Glu Pro Leu Arg Pro | Pro Gln Val Val Cys | Pro Lys Asp His Pro |
| Pro Leu Arg Pro Arg | Gln Val Val Cys Asn | Lys Asp His Pro Leu |
| Leu Arg Pro Arg Cys | Val Val Cys Asn Tyr | Asp His Pro Leu Thr |
| Arg Pro Arg Cys Arg | Val Cys Asn Tyr Arg | His Pro Leu Thr Cys |
| Pro Arg Cys Arg Pro | Cys Asn Tyr Arg Asp | Pro Leu Thr Cys Asp |
| Arg Cys Arg Pro Ile | Asn Tyr Arg Asp Val | Leu Thr Cys Asp Asp |
| Cys Arg Pro Ile Asn | Tyr Arg Asp Val Arg | Thr Cys Asp Asp Pro |
| Arg Pro Ile Asn Ala | Arg Asp Val Arg Phe | Cys Asp Asp Pro Arg |
| Pro Ile Asn Ala Thr | Asp Val Arg Phe Glu | Asp Asp Pro Arg Phe |
| Ile Asn Ala Thr Leu | Val Arg Phe Glu Ser | Asp Pro Arg Phe Gln |
| Asn Ala Thr Leu Ala | Arg Phe Glu Ser Ile | Pro Arg Phe Gln Asp |
| Ala Thr Leu Ala Val | Phe Glu Ser Ile Arg | Arg Phe Gln Asp Ser |
| Thr Leu Ala Val Glu | Glu Ser Ile Arg Leu | Phe Gln Asp Ser Ser |
| Leu Ala Val Glu Lys | Ser Ile Arg Leu Pro | Gln Asp Ser Ser Ser |
| Ala Val Glu Lys Glu | Ile Arg Leu Pro Gly | Asp Ser Ser Ser Ser |
| Val Glu Lys Glu Gly | Arg Leu Pro Gly Cys | Ser Ser Ser Ser Lys |
| Glu Lys Glu Gly Cys | Leu Pro Gly Cys Pro | Ser Ser Ser Lys Ala |
| Lys Glu Gly Cys Pro | Pro Gly Cys Pro Arg | Ser Ser Lys Ala Pro |
| Glu Gly Cys Pro Val | Gly Cys Pro Arg Gly | Ser Lys Ala Pro Pro |
| Gly Cys Pro Val Cys | Cys Pro Arg Gly Val | |
| Cys Pro Val Cys Ile | Pro Arg Gly Val Asn | |
| Pro Val Cys Ile Thr | Arg Gly Val Asn Pro | |
| Val Cys Ile Thr Val | Gly Val Asn Pro Val | |
| Cys Ile Thr Val Asn | Val Asn Pro Val Val | |
| Ile Thr Val Asn Thr | Asn Pro Val Val Ser | |
| Thr Val Asn Thr Thr | Pro Val Val Ser Tyr | |
| Val Asn Thr Thr Ile | Val Val Ser Tyr Ala | |
| Asn Thr Thr Ile Cys | Val Ser Tyr Ala Val | |
| Thr Thr Ile Cys Ala | Ser Tyr Ala Val Ala | |

When myeloperoxidase (MPO) (SEQ ID NO:5) is the target self-antigen, the peptide reagent comprises any sequence of 5 to 15 consecutive amino acid residues from anywhere in the amino acid sequence of MPO (SEQ ID NO:5). Table 6 lists amino acid sequences for exemplary peptide reagents consisting of 5 consecutive amino acid residues from MPO (SEQ ID NO: 5). Additional peptide reagents may have a length of up to 15 amino acid residues, comprising any one of the listed 5-amino acid long sequences in Table 6, plus up to a total of 10 additional consecutive amino acid residues from SEQ ID NO:5, that are continuous (from either side within the protein amino sequence) with the 5-amino acid long sequence.

TABLE 6

| | | |
|---|---|---|
| Gly Val Pro Phe Phe | Ala Val Leu Gly Glu | Phe Lys Gln Pro Val |
| Val Pro Phe Phe Ser | Val Leu Gly Glu Val | Lys Gln Pro Val Ala |
| Pro Phe Phe Ser Ser | Leu Gly Glu Val Asp | Gln Pro Val Ala Ala |
| Phe Phe Ser Ser Leu | Gly Glu Val Asp Thr | Pro Val Ala Ala Thr |
| Phe Ser Ser Leu Arg | Glu Val Asp Thr Ser | Val Ala Ala Thr Arg |
| Ser Ser Leu Arg Cys | Val Asp Thr Ser Leu | Ala Ala Thr Arg Thr |
| Ser Leu Arg Cys Met | Asp Thr Ser Leu Val | Ala Thr Arg Thr Ala |
| Leu Arg Cys Met Val | Thr Ser Leu Val Leu | Thr Arg Thr Ala Val |
| Arg Cys Met Val Asp | Ser Leu Val Leu Ser | Arg Thr Ala Val Arg |
| Cys Met Val Asp Leu | Leu Val Leu Ser Ser | Thr Ala Val Arg Ala |
| Met Val Asp Leu Gly | Val Leu Ser Ser Met | Ala Val Arg Ala Ala |
| Val Asp Leu Gly Pro | Leu Ser Ser Met Glu | Val Arg Ala Ala Asp |
| Asp Leu Gly Pro Cys | Ser Ser Met Glu Glu | Arg Ala Ala Asp Tyr |
| Leu Gly Pro Cys Trp | Ser Met Glu Glu Ala | Ala Ala Asp Tyr Leu |
| Gly Pro Cys Trp Ala | Met Glu Glu Ala Lys | Ala Asp Tyr Leu His |
| Pro Cys Trp Ala Gly | Glu Glu Ala Lys Gln | Asp Tyr Leu His Val |
| Cys Trp Ala Gly Gly | Glu Ala Lys Gln Leu | Tyr Leu His Val Ala |
| Trp Ala Gly Gly Leu | Ala Lys Gln Leu Val | Leu His Val Ala Leu |
| Ala Gly Gly Leu Thr | Lys Gln Leu Val Asp | His Val Ala Leu Asp |
| Gly Gly Leu Thr Ala | Gln Leu Val Asp Lys | Val Ala Leu Asp Leu |
| Gly Leu Thr Ala Glu | Leu Val Asp Lys Ala | Ala Leu Asp Leu Leu |
| Leu Thr Ala Glu Met | Val Asp Lys Ala Tyr | Leu Asp Leu Leu Glu |
| Thr Ala Glu Met Lys | Asp Lys Ala Tyr Lys | Asp Leu Leu Glu Arg |
| Ala Glu Met Lys Leu | Lys Ala Tyr Lys Glu | Leu Leu Glu Arg Lys |
| Glu Met Lys Leu Leu | Ala Tyr Lys Glu Arg | Leu Glu Arg Lys Leu |
| Met Lys Leu Leu Leu | Tyr Lys Glu Arg Arg | Glu Arg Lys Leu Arg |
| Lys Leu Leu Leu Ala | Lys Glu Arg Arg Glu | Arg Lys Leu Arg Ser |
| Leu Leu Leu Ala Leu | Glu Arg Arg Glu Ser | Lys Leu Arg Ser Leu |
| Leu Leu Ala Leu Ala | Arg Arg Glu Ser Ile | Leu Arg Ser Leu Trp |
| Leu Ala Leu Ala Gly | Arg Glu Ser Ile Lys | Arg Ser Leu Trp Arg |
| Ala Leu Ala Gly Leu | Glu Ser Ile Lys Gln | Ser Leu Trp Arg Arg |
| Leu Ala Gly Leu Leu | Ser Ile Lys Gln Arg | Leu Trp Arg Arg Pro |
| Ala Gly Leu Leu Ala | Ile Lys Gln Arg Leu | Trp Arg Arg Pro Phe |
| Gly Leu Leu Ala Ile | Lys Gln Arg Leu Arg | Arg Arg Pro Phe Asn |
| Leu Leu Ala Ile Leu | Gln Arg Leu Arg Ser | Arg Pro Phe Asn Val |
| Leu Ala Ile Leu Ala | Arg Leu Arg Ser Gly | Pro Phe Asn Val Thr |
| Ala Ile Leu Ala Thr | Leu Arg Ser Gly Ser | Phe Asn Val Thr Asp |
| Ile Leu Ala Thr Pro | Arg Ser Gly Ser Ala | Asn Val Thr Asp Val |
| Leu Ala Thr Pro Gln | Ser Gly Ser Ala Ser | Val Thr Asp Val Leu |
| Ala Thr Pro Gln Pro | Gly Ser Ala Ser Pro | Thr Asp Val Leu Thr |
| Thr Pro Gln Pro Ser | Ser Ala Ser Pro Met | Asp Val Leu Thr Pro |
| Pro Gln Pro Ser Glu | Ala Ser Pro Met Glu | Val Leu Thr Pro Ala |
| Gln Pro Ser Glu Gly | Ser Pro Met Glu Leu | Leu Thr Pro Ala Gln |
| Pro Ser Glu Gly Ala | Pro Met Glu Leu Leu | Thr Pro Ala Gln Leu |
| Ser Glu Gly Ala Ala | Met Glu Leu Leu Ser | Pro Ala Gln Leu Asn |
| Glu Gly Ala Ala Pro | Glu Leu Leu Ser Tyr | Ala Gln Leu Asn Val |
| Gly Ala Ala Pro Ala | Leu Leu Ser Tyr Phe | Gln Leu Asn Val Leu |
| Ala Ala Pro Ala Val | Leu Ser Tyr Phe Lys | Leu Asn Val Leu Ser |
| Ala Pro Ala Val Leu | Ser Tyr Phe Lys Gln | Asn Val Leu Ser Lys |
| Pro Ala Val Leu Gly | Tyr Phe Lys Gln Pro | Val Leu Ser Lys Ser |
| Leu Ser Lys Ser Ser | Glu Asp Gly Phe Ser | |
| Ser Lys Ser Ser Gly | Asp Gly Phe Ser Leu | |
| Lys Ser Ser Gly Cys | Gly Phe Ser Leu Pro | |
| Ser Ser Gly Cys Ala | Phe Ser Leu Pro Tyr | |
| Ser Gly Cys Ala Tyr | Ser Leu Pro Tyr Gly | |
| Gly Cys Ala Tyr Gln | Leu Pro Tyr Gly Trp | |
| Cys Ala Tyr Gln Asp | Pro Tyr Gly Trp Thr | |
| Ala Tyr Gln Asp Val | Tyr Gly Trp Thr Pro | |
| Tyr Gln Asp Val Gly | Gly Trp Thr Pro Gly | |
| Gln Asp Val Gly Val | Trp Thr Pro Gly Val | |
| Asp Val Gly Val Thr | Thr Pro Gly Val Lys | |
| Val Gly Val Thr Cys | Pro Gly Val Lys Arg | |
| Gly Val Thr Cys Pro | Gly Val Lys Arg Asn | |
| Val Thr Cys Pro Glu | Val Lys Arg Asn Gly | |
| Thr Cys Pro Glu Gln | Lys Arg Asn Gly Phe | |
| Cys Pro Glu Gln Asp | Arg Asn Gly Phe Pro | |
| Pro Glu Gln Asp Lys | Asn Gly Phe Pro Val | |
| Glu Gln Asp Lys Tyr | Gly Phe Pro Val Ala | |
| Gln Asp Lys Tyr Arg | | |
| Asp Lys Tyr Arg Thr | | |
| Lys Tyr Arg Thr Ile | | |
| Tyr Arg Thr Ile Thr | | |
| Arg Thr Ile Thr Gly | | |
| Thr Ile Thr Gly Met | | |
| Ile Thr Gly Met Cys | | |
| Thr Gly Met Cys Asn | | |
| Gly Met Cys Asn Asn | | |
| Met Cys Asn Asn Arg | | |
| Cys Asn Asn Arg Arg | | |
| Asn Asn Arg Arg Ser | | |
| Asn Arg Arg Ser Pro | | |
| Arg Arg Ser Pro Thr | | |
| Arg Ser Pro Thr Leu | | |
| Ser Pro Thr Leu Gly | | |
| Pro Thr Leu Gly Ala | | |
| Thr Leu Gly Ala Ser | | |
| Leu Gly Ala Ser Asn | | |
| Gly Ala Ser Asn Arg | | |
| Ala Ser Asn Arg Ala | | |
| Ser Asn Arg Ala Phe | | |
| Asn Arg Ala Phe Val | | |
| Arg Ala Phe Val Arg | | |
| Ala Phe Val Arg Trp | | |
| Phe Val Arg Trp Leu | | |
| Val Arg Trp Leu Pro | | |
| Arg Trp Leu Pro Ala | | |
| Trp Leu Pro Ala Glu | | |
| Leu Pro Ala Glu Tyr | | |
| Pro Ala Glu Tyr Glu | | |
| Ala Glu Tyr Glu Asp | | |
| Glu Tyr Glu Asp Gly | | |
| Tyr Glu Asp Gly Phe | | |

When prostate specific antigen (PSA) (SEQ ID NO:6) is the target self-antigen, the peptide reagent comprises any sequence of 5 to 15 consecutive amino acid residues from anywhere in the amino acid sequence of PSA (SEQ ID NO: 6). Table 6 lists amino acid sequences for exemplary peptide reagents consisting of 5 consecutive amino acid residues from PSA (SEQ ID NO: 6). Additional peptide reagents may have a length of up to 15 amino acid residues, comprising any one of the listed 5-amino acid long sequences in Table 7, plus up to a total of 10 additional consecutive amino acid residues from SEQ ID NO:6, that are continuous (from either side within the protein amino sequence) with the 5-amino acid long sequence.

TABLE 7

| | | |
|---|---|---|
| Trp Val Pro Val Val | Gly Val Leu Val His | Asp Met Ser Leu Leu |
| Val Pro Val Val Phe | Val Leu Val His Pro | Met Ser Leu Leu Lys |
| Pro Val Val Phe Leu | Leu Val His Pro Gln | Ser Leu Leu Lys Asn |
| Val Val Phe Leu Thr | Val His Pro Gln Trp | Leu Leu Lys Asn Arg |
| Val Phe Leu Thr Leu | His Pro Gln Trp Val | Leu Lys Asn Arg Phe |
| Phe Leu Thr Leu Ser | Pro Gln Trp Val Leu | Lys Asn Arg Phe Leu |
| Leu Thr Leu Ser Val | Gln Trp Val Leu Thr | Asn Arg Phe Leu Arg |
| Thr Leu Ser Val Thr | Trp Val Leu Thr Ala | Arg Phe Leu Arg Pro |
| Leu Ser Val Thr Trp | Val Leu Thr Ala Ala | Phe Leu Arg Pro Gly |
| Ser Val Thr Trp Ile | Leu Thr Ala Ala His | Leu Arg Pro Gly Asp |
| Val Thr Trp Ile Gly | Thr Ala Ala His Cys | Arg Pro Gly Asp Asp |
| Thr Trp Ile Gly Ala | Ala Ala His Cys Ile | Pro Gly Asp Asp Ser |
| Trp Ile Gly Ala Ala | Ala His Cys Ile Arg | Gly Asp Asp Ser Ser |
| Ile Gly Ala Ala Pro | His Cys Ile Arg Asn | Asp Asp Ser Ser His |
| Gly Ala Ala Pro Leu | Cys Ile Arg Asn Lys | Asp Ser Ser His Asp |
| Ala Ala Pro Leu Ile | Ile Arg Asn Lys Ser | Ser Ser His Asp Leu |
| Ala Pro Leu Ile Leu | Arg Asn Lys Ser Val | Ser His Asp Leu Met |
| Pro Leu Ile Leu Ser | Asn Lys Ser Val Ile | His Asp Leu Met Leu |
| Leu Ile Leu Ser Arg | Lys Ser Val Ile Leu | Asp Leu Met Leu Leu |
| Ile Leu Ser Arg Ile | Ser Val Ile Leu Leu | Leu Met Leu Leu Arg |
| Leu Ser Arg Ile Val | Val Ile Leu Leu Gly | Met Leu Leu Arg Leu |
| Ser Arg Ile Val Gly | Ile Leu Leu Gly Arg | Leu Leu Arg Leu Ser |
| Arg Ile Val Gly Gly | Leu Leu Gly Arg His | Leu Arg Leu Ser Glu |
| Ile Val Gly Gly Trp | Leu Gly Arg His Ser | Arg Leu Ser Glu Pro |
| Val Gly Gly Trp Glu | Gly Arg His Ser Leu | Leu Ser Glu Pro Ala |
| Gly Gly Trp Glu Cys | Arg His Ser Leu Phe | Ser Glu Pro Ala Glu |
| Gly Trp Glu Cys Glu | His Ser Leu Phe His | Glu Pro Ala Glu Leu |
| Trp Glu Cys Glu Lys | Ser Leu Phe His Pro | Pro Ala Glu Leu Thr |
| Glu Cys Glu Lys His | Leu Phe His Pro Glu | Ala Glu Leu Thr Asp |
| Cys Glu Lys His Ser | Phe His Pro Glu Asp | Glu Leu Thr Asp Ala |
| Glu Lys His Ser Gln | His Pro Glu Asp Thr | Leu Thr Asp Ala Val |
| Lys His Ser Gln Pro | Pro Glu Asp Thr Gly | Thr Asp Ala Val Lys |
| His Ser Gln Pro Trp | Glu Asp Thr Gly Gln | Asp Ala Val Lys Val |
| Ser Gln Pro Trp Gln | Asp Thr Gly Gln Val | Ala Val Lys Val Met |
| Gln Pro Trp Gln Val | Thr Gly Gln Val Phe | Val Lys Val Met Asp |
| Pro Trp Gln Val Leu | Gly Gln Val Phe Gln | Lys Val Met Asp Leu |
| Trp Gln Val Leu Val | Gln Val Phe Gln Val | Val Met Asp Leu Pro |
| Gln Val Leu Val Ala | Val Phe Gln Val Ser | Met Asp Leu Pro Thr |
| Val Leu Val Ala Ser | Phe Gln Val Ser His | Asp Leu Pro Thr Gln |
| Leu Val Ala Ser Arg | Gln Val Ser His Ser | Leu Pro Thr Gln Glu |
| Val Ala Ser Arg Gly | Val Ser His Ser Phe | Pro Thr Gln Glu Pro |
| Ala Ser Arg Gly Arg | Ser His Ser Phe Pro | Thr Gln Glu Pro Ala |
| Ser Arg Gly Arg Ala | His Ser Phe Pro His | Gln Glu Pro Ala Leu |
| Arg Gly Arg Ala Val | Ser Phe Pro His Pro | Glu Pro Ala Leu Gly |
| Gly Arg Ala Val Cys | Phe Pro His Pro Leu | Pro Ala Leu Gly Thr |
| Arg Ala Val Cys Gly | Pro His Pro Leu Tyr | Ala Leu Gly Thr Thr |
| Ala Val Cys Gly Gly | His Pro Leu Tyr Asp | Leu Gly Thr Thr Cys |
| Val Cys Gly Gly Val | Pro Leu Tyr Asp Met | Gly Thr Thr Cys Tyr |
| Cys Gly Gly Val Leu | Leu Tyr Asp Met Ser | Thr Thr Cys Tyr Ala |
| Gly Gly Val Leu Val | Tyr Asp Met Ser Leu | Thr Cys Tyr Ala Ser |
| Cys Tyr Ala Ser Gly | Gly Gly Lys Ser Thr | Thr Ile Val Ala Asn |
| Tyr Ala Ser Gly Trp | Gly Lys Ser Thr Cys | Ile Val Ala Asn Pro |
| Ala Ser Gly Trp Gly | Lys Ser Thr Cys Ser | |
| Ser Gly Trp Gly Ser | Ser Thr Cys Ser Gly | |
| Gly Trp Gly Ser Ile | Thr Cys Ser Gly Asp | |
| Trp Gly Ser Ile Glu | Cys Ser Gly Asp Ser | |
| Gly Ser Ile Glu Pro | Ser Gly Asp Ser Gly | |
| Ser Ile Glu Pro Glu | Gly Asp Ser Gly Gly | |
| Ile Glu Pro Glu Glu | Asp Ser Gly Gly Pro | |
| Glu Pro Glu Glu Phe | Ser Gly Gly Pro Leu | |
| Pro Glu Glu Phe Leu | Gly Gly Pro Leu Val | |
| Glu Glu Phe Leu Thr | Gly Pro Leu Val Cys | |
| Glu Phe Leu Thr Pro | Pro Leu Val Cys Asn | |
| Phe Leu Thr Pro Lys | Leu Val Cys Asn Gly | |
| Leu Thr Pro Lys Lys | Val Cys Asn Gly Val | |
| Thr Pro Lys Lys Leu | Cys Asn Gly Val Leu | |
| Pro Lys Lys Leu Gln | Asn Gly Val Leu Gln | |
| Lys Lys Leu Gln Cys | Gly Val Leu Gln Gly | |
| Lys Leu Gln Cys Val | Val Leu Gln Gly Ile | |
| Leu Gln Cys Val Asp | Leu Gln Gly Ile Thr | |
| Gln Cys Val Asp Leu | Gln Gly Ile Thr Ser | |
| Cys Val Asp Leu His | Gly Ile Thr Ser Trp | |
| Val Asp Leu His Val | Ile Thr Ser Trp Gly | |
| Asp Leu His Val Ile | Thr Ser Trp Gly Ser | |
| Leu His Val Ile Ser | Ser Trp Gly Ser Glu | |
| His Val Ile Ser Asn | Trp Gly Ser Glu Pro | |
| Val Ile Ser Asn Asp | Gly Ser Glu Pro Cys | |
| Ile Ser Asn Asp Val | Ser Glu Pro Cys Ala | |
| Ser Asn Asp Val Cys | Glu Pro Cys Ala Leu | |
| Asn Asp Val Cys Ala | Pro Cys Ala Leu Pro | |
| Asp Val Cys Ala Gln | Cys Ala Leu Pro Glu | |
| Val Cys Ala Gln Val | Ala Leu Pro Glu Arg | |
| Cys Ala Gln Val His | Leu Pro Glu Arg Pro | |
| Ala Gln Val His Pro | Pro Glu Arg Pro Ser | |
| Gln Val His Pro Gln | Glu Arg Pro Ser Leu | |
| Val His Pro Gln Lys | Arg Pro Ser Leu Tyr | |
| His Pro Gln Lys Val | Pro Ser Leu Tyr Thr | |
| Pro Gln Lys Val Thr | Ser Leu Tyr Thr Lys | |
| Gln Lys Val Thr Lys | Leu Tyr Thr Lys Val | |
| Lys Val Thr Lys Phe | Tyr Thr Lys Val Val | |
| Val Thr Lys Phe Met | Thr Lys Val Val His | |
| Thr Lys Phe Met Leu | Lys Val Val His Tyr | |
| Lys Phe Met Leu Cys | Val Val His Tyr Arg | |
| Phe Met Leu Cys Ala | Val His Tyr Arg Lys | |
| Met Leu Cys Ala Gly | His Tyr Arg Lys Trp | |
| Leu Cys Ala Gly Arg | Tyr Arg Lys Trp Ile | |
| Cys Ala Gly Arg Trp | Arg Lys Trp Ile Lys | |
| Ala Gly Arg Trp Thr | Lys Trp Ile Lys Asp | |
| Gly Arg Trp Thr Gly | Trp Ile Lys Asp Thr | |
| Arg Trp Thr Gly Gly | Ile Lys Asp Thr Ile | |
| Trp Thr Gly Gly Lys | Lys Asp Thr Ile Val | |
| Thr Gly Gly Lys Ser | Asp Thr Ile Val Ala | |

When human B-type natriuretic peptide (hBNP) (SEQ ID NO:7) is the target self-antigen, the peptide reagent comprises any sequence of 5 to 15 consecutive amino acid residues from anywhere in the amino acid sequence of hBNP (SEQ ID NO:7). Table 8 lists amino acid sequences for exemplary peptide reagents consisting of 5 consecutive amino acid residues from hBNP (SEQ ID NO: 7). Additional peptide reagents may have a length of up to 15 amino acid residues, comprising any one of the listed 5-amino acid long sequences in Table 8, plus up to a total of 10 additional consecutive amino acid residues from SEQ ID NO:7, that are continuous (from either side within the protein amino sequence) with the 5-amino acid long sequence.

TABLE 8

| | | |
|---|---|---|
| Asp Pro Gln Thr Ala | Gly Lys Leu Ser Glu | Arg Ser Pro Lys Met |
| Pro Gln Thr Ala Pro | Lys Leu Ser Glu Leu | Ser Pro Lys Met Val |
| Gln Thr Ala Pro Ser | Leu Ser Glu Leu Gln | Pro Lys Met Val Gln |
| Thr Ala Pro Ser Arg | Ser Glu Leu Gln Val | Lys Met Val Gln Gly |
| Ala Pro Ser Arg Ala | Glu Leu Gln Val Glu | Met Val Gln Gly Ser |
| Pro Ser Arg Ala Leu | Leu Gln Val Glu Gln | Val Gln Gly Ser Gly |
| Ser Arg Ala Leu Leu | Gln Val Glu Gln Thr | Gln Gly Ser Gly Cys |
| Arg Ala Leu Leu Leu | Val Glu Gln Thr Ser | Gly Ser Gly Cys Phe |
| Ala Leu Leu Leu Leu | Glu Gln Thr Ser Leu | Ser Gly Cys Phe Gly |
| Leu Leu Leu Leu Leu | Gln Thr Ser Leu Glu | Gly Cys Phe Gly Arg |
| Leu Leu Leu Leu Phe | Thr Ser Leu Glu Pro | Cys Phe Gly Arg Lys |
| Leu Leu Leu Phe Leu | Ser Leu Glu Pro Leu | Phe Gly Arg Lys Met |
| Leu Leu Phe Leu His | Leu Glu Pro Leu Gln | Gly Arg Lys Met Asp |
| Leu Phe Leu His Leu | Glu Pro Leu Gln Glu | Arg Lys Met Asp Arg |
| Phe Leu His Leu Ala | Pro Leu Gln Glu Ser | Lys Met Asp Arg Ile |
| Leu His Leu Ala Phe | Leu Gln Glu Ser Pro | Met Asp Arg Ile Ser |
| His Leu Ala Phe Leu | Gln Glu Ser Pro Arg | Asp Arg Ile Ser Ser |
| Leu Ala Phe Leu Gly | Glu Ser Pro Arg Pro | Arg Ile Ser Ser Ser |
| Ala Phe Leu Gly Gly | Ser Pro Arg Pro Thr | Ile Ser Ser Ser Ser |
| Phe Leu Gly Gly Arg | Pro Arg Pro Thr Gly | Ser Ser Ser Ser Gly |
| Leu Gly Gly Arg Ser | Arg Pro Thr Gly Val | Ser Ser Ser Gly Leu |
| Gly Gly Arg Ser His | Pro Thr Gly Val Trp | Ser Ser Gly Leu Gly |
| Gly Arg Ser His Pro | Thr Gly Val Trp Lys | Ser Gly Leu Gly Cys |
| Arg Ser His Pro Leu | Gly Val Trp Lys Ser | Gly Leu Gly Cys Lys |
| Ser His Pro Leu Gly | Val Trp Lys Ser Arg | Leu Gly Cys Lys Val |
| His Pro Leu Gly Ser | Trp Lys Ser Arg Glu | Gly Cys Lys Val Leu |
| Pro Leu Gly Ser Pro | Lys Ser Arg Glu Val | Cys Lys Val Leu Arg |
| Leu Gly Ser Pro Gly | Ser Arg Glu Val Ala | Lys Val Leu Arg Arg |
| Gly Ser Pro Gly Ser | Arg Glu Val Ala Thr | Val Leu Arg Arg His |
| Ser Pro Gly Ser Ala | Glu Val Ala Thr Glu | |
| Pro Gly Ser Ala Ser | Val Ala Thr Glu Gly | |
| Gly Ser Ala Ser Asp | Ala Thr Glu Gly Ile | |
| Ser Ala Ser Asp Leu | Thr Glu Gly Ile Arg | |
| Ala Ser Asp Leu Glu | Glu Gly Ile Arg Gly | |
| Ser Asp Leu Glu Thr | Gly Ile Arg Gly His | |
| Asp Leu Glu Thr Ser | Ile Arg Gly His Arg | |
| Leu Glu Thr Ser Gly | Arg Gly His Arg Lys | |
| Glu Thr Ser Gly Leu | Gly His Arg Lys Met | |
| Thr Ser Gly Leu Gln | His Arg Lys Met Val | |
| Ser Gly Leu Gln Glu | Arg Lys Met Val Leu | |
| Gly Leu Gln Glu Gln | Lys Met Val Leu Tyr | |
| Leu Gln Glu Gln Arg | Met Val Leu Tyr Thr | |
| Gln Glu Gln Arg Asn | Val Leu Tyr Thr Leu | |
| Glu Gln Arg Asn His | Leu Tyr Thr Leu Arg | |
| Gln Arg Asn His Leu | Tyr Thr Leu Arg Ala | |
| Arg Asn His Leu Gln | Thr Leu Arg Ala Pro | |
| Asn His Leu Gln Gly | Leu Arg Ala Pro Arg | |
| His Leu Gln Gly Lys | Arg Ala Pro Arg Ser | |
| Leu Gln Gly Lys Leu | Ala Pro Arg Ser Pro | |
| Gln Gly Lys Leu Ser | Pro Arg Ser Pro Lys | |

When myosin light chain 2 (SEQ ID NO:8) is the target self-antigen, the peptide reagent comprises any sequence of 5 to 15 consecutive amino acid residues from anywhere in the amino acid sequence of myosin light chain 2 (SEQ ID NO:8). Table 9 lists amino acid sequences for exemplary peptide reagents consisting of 5 consecutive amino acid residues from myosin light chain 2 (SEQ ID NO: 8). Additional peptide reagents may have a length of up to 15 amino acid residues, comprising any one of the listed 5-amino acid long sequences in Table 9, plus up to a total of 10 additional consecutive amino acid residues from SEQ ID NO:8, that are continuous (from either side within the protein amino sequence) with the 5-amino acid long sequence.

TABLE 9

| | | |
|---|---|---|
| Ala Pro Lys Lys Ala | Thr Phe Ala Ala Leu | Ala Phe Lys Val Phe |
| Pro Lys Lys Ala Lys | Phe Ala Ala Leu Gly | Phe Lys Val Phe Asp |
| Lys Lys Ala Lys Lys | Ala Ala Leu Gly Arg | Lys Val Phe Asp Pro |
| Lys Ala Lys Lys Arg | Ala Leu Gly Arg Val | Val Phe Asp Pro Glu |
| Ala Lys Lys Arg Ala | Leu Gly Arg Val Asn | Phe Asp Pro Glu Gly |
| Lys Lys Arg Ala Gly | Gly Arg Val Asn Val | Asp Pro Glu Gly Lys |
| Lys Arg Ala Gly Gly | Arg Val Asn Val Lys | Pro Glu Gly Lys Gly |
| Arg Ala Gly Gly Ala | Val Asn Val Lys Asn | Glu Gly Lys Gly Val |
| Ala Gly Gly Ala Asn | Asn Val Lys Asn Glu | Gly Lys Gly Val Leu |
| Gly Gly Ala Asn Ser | Val Lys Asn Glu Glu | Lys Gly Val Leu Lys |
| Gly Ala Asn Ser Asn | Lys Asn Glu Glu Ile | Gly Val Leu Lys Ala |
| Ala Asn Ser Asn Val | Asn Glu Glu Ile Asp | Val Leu Lys Ala Asp |
| Asn Ser Asn Val Phe | Glu Glu Ile Asp Glu | Leu Lys Ala Asp Tyr |
| Ser Asn Val Phe Ser | Glu Ile Asp Glu Met | Lys Ala Asp Tyr Val |
| Asn Val Phe Ser Met | Ile Asp Glu Met Ile | Ala Asp Tyr Val Arg |
| Val Phe Ser Met Phe | Asp Glu Met Ile Lys | Asp Tyr Val Arg Glu |
| Phe Ser Met Phe Glu | Glu Met Ile Lys Glu | Tyr Val Arg Glu Met |
| Ser Met Phe Glu Gln | Met Ile Lys Glu Ala | Val Arg Glu Met Leu |
| Met Phe Glu Gln Thr | Ile Lys Glu Ala Pro | Arg Glu Met Leu Thr |
| Phe Glu Gln Thr Gln | Lys Glu Ala Pro Gly | Glu Met Leu Thr Thr |
| Glu Gln Thr Gln Ile | Glu Ala Pro Gly Pro | Met Leu Thr Thr Gln |
| Gln Thr Gln Ile Gln | Ala Pro Gly Pro Ile | Leu Thr Thr Gln Ala |
| Thr Gln Ile Gln Glu | Pro Gly Pro Ile Asn | Thr Thr Gln Ala Glu |
| Gln Ile Gln Glu Phe | Gly Pro Ile Asn Phe | Thr Gln Ala Glu Arg |
| Ile Gln Glu Phe Lys | Pro Ile Asn Phe Thr | Gln Ala Glu Arg Phe |
| Gln Glu Phe Lys Glu | Ile Asn Phe Thr Val | Ala Glu Arg Phe Ser |
| Glu Phe Lys Glu Ala | Asn Phe Thr Val Phe | Glu Arg Phe Ser Lys |
| Phe Lys Glu Ala Phe | Phe Thr Val Phe Leu | Arg Phe Ser Lys Glu |
| Lys Glu Ala Phe Thr | Thr Val Phe Leu Thr | Phe Ser Lys Glu Glu |
| Glu Ala Phe Thr Ile | Val Phe Leu Thr Met | Ser Lys Glu Glu Val |
| Ala Phe Thr Ile Met | Phe Leu Thr Met Phe | Lys Glu Glu Val Asp |
| Phe Thr Ile Met Asp | Leu Thr Met Phe Gly | Glu Glu Val Asp Gln |
| Thr Ile Met Asp Gln | Thr Met Phe Gly Glu | Glu Val Asp Gln Met |
| Ile Met Asp Gln Asn | Met Phe Gly Glu Lys | Val Asp Gln Met Phe |
| Met Asp Gln Asn Arg | Phe Gly Glu Lys Leu | Asp Gln Met Phe Ala |
| Asp Gln Asn Arg Asp | Gly Glu Lys Leu Lys | Gln Met Phe Ala Ala |
| Gln Asn Arg Asp Gly | Glu Lys Leu Lys Gly | Met Phe Ala Ala Phe |
| Asn Arg Asp Gly Phe | Lys Leu Lys Gly Ala | Phe Ala Ala Phe Pro |
| Arg Asp Gly Phe Ile | Leu Lys Gly Ala Asp | Ala Ala Phe Pro Pro |
| Asp Gly Phe Ile Asp | Lys Gly Ala Asp Pro | Ala Phe Pro Pro Asp |
| Gly Phe Ile Asp Lys | Gly Ala Asp Pro Glu | Phe Pro Pro Asp Val |
| Phe Ile Asp Lys Asn | Ala Asp Pro Glu Glu | Pro Pro Asp Val Thr |
| Ile Asp Lys Asn Asp | Asp Pro Glu Glu Thr | Pro Asp Val Thr Gly |
| Asp Lys Asn Asp Leu | Pro Glu Glu Thr Ile | Asp Val Thr Gly Asn |
| Lys Asn Asp Leu Arg | Glu Glu Thr Ile Leu | Val Thr Gly Asn Leu |
| Asn Asp Leu Arg Asp | Glu Thr Ile Leu Asn | Thr Gly Asn Leu Asp |
| Asp Leu Arg Asp Thr | Thr Ile Leu Asn Ala | Gly Asn Leu Asp Tyr |
| Leu Arg Asp Thr Phe | Ile Leu Asn Ala Phe | Asn Leu Asp Tyr Lys |
| Arg Asp Thr Phe Ala | Leu Asn Ala Phe Lys | Leu Asp Tyr Lys Asn |
| Asp Thr Phe Ala Ala | Asn Ala Phe Lys Val | Asp Tyr Lys Asn Leu |
| Tyr Lys Asn Leu Val | | |
| Lys Asn Leu Val His | | |
| Asn Leu Val His Ile | | |
| Leu Val His Ile Ile | | |
| Val His Ile Ile Thr | | |
| His Ile Ile Thr His | | |
| Ile Ile Thr His Gly | | |
| Ile Thr His Gly Glu | | |
| Thr His Gly Glu Glu | | |
| His Gly Glu Glu Lys | | |
| Gly Glu Glu Lys Asp | | |

When myosin-6 (SEQ ID NO:9) is the target self-antigen, the peptide reagent comprises any sequence of 5 to 15 consecutive amino acid residues from anywhere in the amino acid sequence of myosin-6 (SEQ ID NO:9). Table 10 lists amino acid sequences for exemplary peptide reagents consisting of 5 consecutive amino acid residues from myosin-6 (SEQ ID NO: 9). Additional peptide reagents may have a length of up to 15 amino acid residues, comprising any one of the listed 5-amino acid long sequences in Table 10, plus up to a total of 10 additional consecutive amino acid residues from SEQ ID NO:9, that are continuous (from either side within the protein amino sequence) with the 5-amino acid long sequence.

TABLE 10

| | | |
|---|---|---|
| Thr Asp Ala Gln Met | Leu Ser Arg Glu Gly | Leu Phe Asn Leu Lys |
| Asp Ala Gln Met Ala | Ser Arg Glu Gly Gly | Phe Asn Leu Lys Glu |
| Ala Gln Met Ala Asp | Arg Glu Gly Gly Lys | Asn Leu Lys Glu Arg |
| Gln Met Ala Asp Phe | Glu Gly Gly Lys Val | Leu Lys Glu Arg Tyr |
| Met Ala Asp Phe Gly | Gly Gly Lys Val Ile | Lys Glu Arg Tyr Ala |
| Ala Asp Phe Gly Ala | Gly Lys Val Ile Ala | Glu Arg Tyr Ala Ala |
| Asp Phe Gly Ala Ala | Lys Val Ile Ala Glu | Arg Tyr Ala Ala Trp |
| Phe Gly Ala Ala Ala | Val Ile Ala Glu Thr | Tyr Ala Ala Trp Met |
| Gly Ala Ala Ala Gln | Ile Ala Glu Thr Glu | Ala Ala Trp Met Ile |
| Ala Ala Ala Gln Tyr | Ala Glu Thr Glu Asn | Ala Trp Met Ile Tyr |
| Ala Ala Gln Tyr Leu | Glu Thr Glu Asn Gly | Trp Met Ile Tyr Thr |
| Ala Gln Tyr Leu Arg | Thr Glu Asn Gly Lys | Met Ile Tyr Thr Tyr |
| Gln Tyr Leu Arg Lys | Glu Asn Gly Lys Thr | Ile Tyr Thr Tyr Ser |
| Tyr Leu Arg Lys Ser | Asn Gly Lys Thr Val | Tyr Thr Tyr Ser Gly |
| Leu Arg Lys Ser Glu | Gly Lys Thr Val Thr | Thr Tyr Ser Gly Leu |
| Arg Lys Ser Glu Lys | Lys Thr Val Thr Val | Tyr Ser Gly Leu Phe |
| Lys Ser Glu Lys Glu | Thr Val Thr Val Lys | Ser Gly Leu Phe Cys |
| Ser Glu Lys Glu Arg | Val Thr Val Lys Glu | Gly Leu Phe Cys Val |
| Glu Lys Glu Arg Leu | Thr Val Lys Glu Asp | Leu Phe Cys Val Thr |
| Lys Glu Arg Leu Glu | Val Lys Glu Asp Gln | Phe Cys Val Thr Val |
| Glu Arg Leu Glu Ala | Lys Glu Asp Gln Val | Cys Val Thr Val Asn |
| Arg Leu Glu Ala Gln | Glu Asp Gln Val Leu | Val Thr Val Asn Pro |
| Leu Glu Ala Gln Thr | Asp Gln Val Leu Gln | Thr Val Asn Pro Tyr |
| Glu Ala Gln Thr Arg | Gln Val Leu Gln Gln | Val Asn Pro Tyr Lys |
| Ala Gln Thr Arg Pro | Val Leu Gln Gln Asn | Asn Pro Tyr Lys Trp |
| Gln Thr Arg Pro Phe | Leu Gln Gln Asn Pro | Pro Tyr Lys Trp Leu |
| Thr Arg Pro Phe Asp | Gln Gln Asn Pro Pro | Tyr Lys Trp Leu Pro |
| Arg Pro Phe Asp Ile | Gln Asn Pro Pro Lys | Lys Trp Leu Pro Val |
| Pro Phe Asp Ile Arg | Asn Pro Pro Lys Phe | Trp Leu Pro Val Tyr |
| Phe Asp Ile Arg Thr | Pro Pro Lys Phe Asp | Leu Pro Val Tyr Asn |
| Asp Ile Arg Thr Glu | Pro Lys Phe Asp Lys | Pro Val Tyr Asn Ala |
| Ile Arg Thr Glu Cys | Lys Phe Asp Lys Ile | Val Tyr Asn Ala Glu |
| Arg Thr Glu Cys Phe | Phe Asp Lys Ile Gln | Tyr Asn Ala Glu Val |
| Thr Glu Cys Phe Val | Asp Lys Ile Gln Asp | Asn Ala Glu Val Val |
| Glu Cys Phe Val Pro | Lys Ile Gln Asp Met | Ala Glu Val Val Ala |
| Cys Phe Val Pro Asp | Ile Gln Asp Met Ala | Glu Val Val Ala Ala |
| Phe Val Pro Asp Asp | Gln Asp Met Ala Met | Val Val Ala Ala Tyr |
| Val Pro Asp Asp Lys | Asp Met Ala Met Leu | Val Ala Ala Tyr Arg |
| Pro Asp Asp Lys Glu | Met Ala Met Leu Thr | Ala Ala Tyr Arg Gly |
| Asp Asp Lys Glu Glu | Ala Met Leu Thr Phe | Ala Tyr Arg Gly Lys |
| Asp Lys Glu Glu Phe | Met Leu Thr Phe Leu | Tyr Arg Gly Lys Lys |
| Lys Glu Glu Phe Val | Leu Thr Phe Leu His | Arg Gly Lys Lys Arg |
| Glu Glu Phe Val Lys | Thr Phe Leu His Glu | Gly Lys Lys Arg Ser |
| Glu Phe Val Lys Ala | Phe Leu His Glu Pro | Lys Lys Arg Ser Glu |
| Phe Val Lys Ala Lys | Leu His Glu Pro Ala | Lys Arg Ser Glu Ala |
| Val Lys Ala Lys Ile | His Glu Pro Ala Val | Arg Ser Glu Ala Pro |
| Lys Ala Lys Ile Leu | Glu Pro Ala Val Leu | Ser Glu Ala Pro Pro |
| Ala Lys Ile Leu Ser | Pro Ala Val Leu Phe | Glu Ala Pro Pro His |
| Lys Ile Leu Ser Arg | Ala Val Leu Phe Asn | Ala Pro Pro His Ile |
| Ile Leu Ser Arg Glu | Val Leu Phe Asn Leu | Pro Pro His Ile Phe |
| Pro His Ile Phe Ser | Arg Gly Lys Lys Asp | Thr Gly Lys Leu Ala |
| His Ile Phe Ser Ile | Gly Lys Lys Asp Asn | Gly Lys Leu Ala Ser |
| Ile Phe Ser Ile Ser | Lys Lys Asp Asn Ala | Lys Leu Ala Ser Ala |
| Phe Ser Ile Ser Asp | Lys Asp Asn Ala Asn | Leu Ala Ser Ala Asp |
| Ser Ile Ser Asp Asn | Asp Asn Ala Asn Ala | Ala Ser Ala Asp Ile |
| Ile Ser Asp Asn Ala | Asn Ala Asn Ala Asn | Ser Ala Asp Ile Glu |
| Ser Asp Asn Ala Tyr | Ala Asn Ala Asn Lys | Ala Asp Ile Glu Thr |
| Asp Asn Ala Tyr Gln | Asn Ala Asn Lys Gly | Asp Ile Glu Thr Tyr |
| Asn Ala Tyr Gln Tyr | Ala Asn Lys Gly Thr | Ile Glu Thr Tyr Leu |
| Ala Tyr Gln Tyr Met | Asn Lys Gly Thr Leu | Glu Thr Tyr Leu Leu |
| Tyr Gln Tyr Met Leu | Lys Gly Thr Leu Glu | Thr Tyr Leu Leu Glu |
| Gln Tyr Met Leu Thr | Gly Thr Leu Glu Asp | Tyr Leu Leu Glu Lys |
| Tyr Met Leu Thr Asp | Thr Leu Glu Asp Gln | Leu Leu Glu Lys Ser |
| Met Leu Thr Asp Arg | Leu Glu Asp Gln Ile | Leu Glu Lys Ser Arg |
| Leu Thr Asp Arg Glu | Glu Asp Gln Ile Ile | Glu Lys Ser Arg Val |
| Thr Asp Arg Glu Asn | Asp Gln Ile Ile Gln | Lys Ser Arg Val Ile |
| Asp Arg Glu Asn Gln | Gln Ile Ile Gln Ala | Ser Arg Val Ile Phe |
| Arg Glu Asn Gln Ser | Ile Ile Gln Ala Asn | Arg Val Ile Phe Gln |
| Glu Asn Gln Ser Ile | Ile Gln Ala Asn Pro | Val Ile Phe Gln Leu |
| Asn Gln Ser Ile Leu | Gln Ala Asn Pro Ala | Ile Phe Gln Leu Lys |
| Gln Ser Ile Leu Ile | Ala Asn Pro Ala Leu | Phe Gln Leu Lys Ala |
| Ser Ile Leu Ile Thr | Asn Pro Ala Leu Glu | Gln Leu Lys Ala Glu |
| Ile Leu Ile Thr Gly | Pro Ala Leu Glu Ala | Leu Lys Ala Glu Arg |
| Leu Ile Thr Gly Glu | Ala Leu Glu Ala Phe | Lys Ala Glu Arg Asn |
| Ile Thr Gly Glu Ser | Leu Glu Ala Phe Gly | Ala Glu Arg Asn Tyr |
| Thr Gly Glu Ser Gly | Glu Ala Phe Gly Asn | Glu Arg Asn Tyr His |
| Gly Glu Ser Gly Ala | Ala Phe Gly Asn Ala | Arg Asn Tyr His Ile |
| Glu Ser Gly Ala Gly | Phe Gly Asn Ala Lys | Asn Tyr His Ile Phe |
| Ser Gly Ala Gly Lys | Gly Asn Ala Lys Thr | Tyr His Ile Phe Tyr |
| Gly Ala Gly Lys Thr | Asn Ala Lys Thr Val | His Ile Phe Tyr Gln |
| Ala Gly Lys Thr Val | Ala Lys Thr Val Arg | Ile Phe Tyr Gln Ile |
| Gly Lys Thr Val Asn | Lys Thr Val Arg Asn | Phe Tyr Gln Ile Leu |
| Lys Thr Val Asn Thr | Thr Val Arg Asn Asp | Tyr Gln Ile Leu Ser |
| Thr Val Asn Thr Lys | Val Arg Asn Asp Asn | Gln Ile Leu Ser Asn |
| Val Asn Thr Lys Arg | Arg Asn Asp Asn Ser | Ile Leu Ser Asn Lys |
| Asn Thr Lys Arg Val | Asn Asp Asn Ser Ser | Leu Ser Asn Lys Lys |
| Thr Lys Arg Val Ile | Asp Asn Ser Ser Arg | Ser Asn Lys Lys Pro |
| Lys Arg Val Ile Gln | Asn Ser Ser Arg Phe | Asn Lys Lys Pro Glu |
| Arg Val Ile Gln Tyr | Ser Ser Arg Phe Gly | Lys Lys Pro Glu Leu |
| Val Ile Gln Tyr Phe | Ser Arg Phe Gly Lys | Lys Pro Glu Leu Leu |
| Ile Gln Tyr Phe Ala | Arg Phe Gly Lys Phe | Pro Glu Leu Leu Asp |
| Gln Tyr Phe Ala Ser | Phe Gly Lys Phe Ile | Glu Leu Leu Asp Met |
| Tyr Phe Ala Ser Ile | Gly Lys Phe Ile Arg | Leu Leu Asp Met Leu |
| Phe Ala Ser Ile Ala | Lys Phe Ile Arg Ile | Leu Asp Met Leu Leu |
| Ala Ser Ile Ala Ala | Phe Ile Arg Ile His | Asp Met Leu Leu Val |
| Ser Ile Ala Ala Ile | Ile Arg Ile His Phe | Met Leu Leu Val Thr |
| Ile Ala Ala Ile Gly | Arg Ile His Phe Gly | Leu Leu Val Thr Asn |
| Ala Ala Ile Gly Asp | Ile His Phe Gly Ala | Leu Val Thr Asn Asn |
| Ala Ile Gly Asp Arg | His Phe Gly Ala Thr | Val Thr Asn Asn Pro |
| Ile Gly Asp Arg Gly | Phe Gly Ala Thr Gly | Thr Asn Asn Pro Tyr |
| Gly Asp Arg Gly Lys | Gly Ala Thr Gly Lys | Asn Asn Pro Tyr Asp |
| Asp Arg Gly Lys Lys | Ala Thr Gly Lys Leu | Asn Pro Tyr Asp Tyr |
| Pro Tyr Asp Tyr Ala | Tyr Gly Asn Met Lys | Val Thr Lys Gly Gln |
| Tyr Asp Tyr Ala Phe | Gly Asn Met Lys Phe | Thr Lys Gly Gln Ser |
| Asp Tyr Ala Phe Val | Asn Met Lys Phe Lys | Lys Gly Gln Ser Val |
| Tyr Ala Phe Val Ser | Met Lys Phe Lys Gln | Gly Gln Ser Val Gln |
| Ala Phe Val Ser Gln | Lys Phe Lys Gln Lys | Gln Ser Val Gln Gln |
| Phe Val Ser Gln Gly | Phe Lys Gln Lys Gln | Ser Val Gln Gln Val |
| Val Ser Gln Gly Glu | Lys Gln Lys Gln Arg | Val Gln Gln Val Tyr |
| Ser Gln Gly Glu Val | Gln Lys Gln Arg Glu | Gln Gln Val Tyr Tyr |
| Gln Gly Glu Val Ser | Lys Gln Arg Glu Glu | Gln Val Tyr Tyr Ser |
| Gly Glu Val Ser Val | Gln Arg Glu Glu Gln | Val Tyr Tyr Ser Ile |
| Glu Val Ser Val Ala | Arg Glu Glu Gln Ala | Tyr Tyr Ser Ile Gly |
| Val Ser Val Ala Ser | Glu Glu Gln Ala Glu | Tyr Ser Ile Gly Ala |
| Ser Val Ala Ser Ile | Glu Gln Ala Glu Pro | Ser Ile Gly Ala Leu |
| Val Ala Ser Ile Asp | Gln Ala Glu Pro Asp | Ile Gly Ala Leu Ala |
| Ala Ser Ile Asp Asp | Ala Glu Pro Asp Gly | Gly Ala Leu Ala Lys |
| Ser Ile Asp Asp Ser | Glu Pro Asp Gly Thr | Ala Leu Ala Lys Ala |
| Ile Asp Asp Ser Glu | Pro Asp Gly Thr Glu | Leu Ala Lys Ala Val |
| Asp Asp Ser Glu Glu | Asp Gly Thr Glu Asp | Ala Lys Ala Val Tyr |
| Asp Ser Glu Glu Leu | Gly Thr Glu Asp Ala | |
| Ser Glu Glu Leu Met | Thr Glu Asp Ala Asp | |
| Glu Glu Leu Met Ala | Glu Asp Ala Asp Lys | |
| Glu Leu Met Ala Thr | Asp Ala Asp Lys Ser | |
| Leu Met Ala Thr Asp | Ala Asp Lys Ser Ala | |
| Met Ala Thr Asp Ser | Asp Lys Ser Ala Tyr | |
| Ala Thr Asp Ser Ala | Lys Ser Ala Tyr Leu | |
| Thr Asp Ser Ala Phe | Ser Ala Tyr Leu Met | |
| Asp Ser Ala Phe Asp | Ala Tyr Leu Met Gly | |
| Ser Ala Phe Asp Val | Tyr Leu Met Gly Leu | |
| Ala Phe Asp Val Leu | Leu Met Gly Leu Asn | |
| Phe Asp Val Leu Gly | Met Gly Leu Asn Ser | |
| Asp Val Leu Gly Phe | Gly Leu Asn Ser Ala | |
| Val Leu Gly Phe Thr | Leu Asn Ser Ala Asp | |
| Leu Gly Phe Thr Ser | Asn Ser Ala Asp Leu | |
| Gly Phe Thr Ser Glu | Ser Ala Asp Leu Leu | |
| Phe Thr Ser Glu Glu | Ala Asp Leu Leu Lys | |
| Thr Ser Glu Glu Lys | Asp Leu Leu Lys Gly | |
| Ser Glu Glu Lys Ala | Leu Leu Lys Gly Leu | |
| Glu Glu Lys Ala Gly | Leu Lys Gly Leu Cys | |
| Glu Lys Ala Gly Val | Lys Gly Leu Cys His | |
| Lys Ala Gly Val Tyr | Gly Leu Cys His Pro | |
| Ala Gly Val Tyr Lys | Leu Cys His Pro Arg | |
| Gly Val Tyr Lys Leu | Cys His Pro Arg Val | |
| Val Tyr Lys Leu Thr | His Pro Arg Val Lys | |
| Tyr Lys Leu Thr Gly | Pro Arg Val Lys Val | |
| Lys Leu Thr Gly Ala | Arg Val Lys Val Gly | |
| Leu Thr Gly Ala Ile | Val Lys Val Gly Asn | |
| Thr Gly Ala Ile Met | Lys Val Gly Asn Glu | |
| Gly Ala Ile Met His | Val Gly Asn Glu Tyr | |
| Ala Ile Met His Tyr | Gly Asn Glu Tyr Val | |
| Ile Met His Tyr Gly | Asn Glu Tyr Val Thr | |
| Met His Tyr Gly Asn | Glu Tyr Val Thr Lys | |
| His Tyr Gly Asn Met | Tyr Val Thr Lys Gly | |

When myosin-7 (SEQ ID NO:10) is the target self-antigen, the peptide reagent comprises any sequence of 5 to 15 consecutive amino acid residues from anywhere in the amino acid sequence of myosin-7 (SEQ ID NO:10). Table 11 lists amino acid sequences for exemplary peptide reagents consisting of 5 consecutive amino acid residues from myosin-7 (SEQ ID NO: 10). Additional peptide reagents may have a length of up to 15 amino acid residues, comprising any one of the listed 5-amino acid long sequences in Table 11, plus up to a total of 10 additional consecutive amino acid residues from SEQ ID NO:10, that are continuous (from either side within the protein amino sequence) with the 5-amino acid long sequence.

TABLE 11

| | | |
|---|---|---|
| Gly Asp Ser Glu Met | Val Ser Arg Glu Gly | Leu Tyr Asn Leu Lys |
| Asp Ser Glu Met Ala | Ser Arg Glu Gly Gly | Tyr Asn Leu Lys Asp |
| Ser Glu Met Ala Val | Arg Glu Gly Gly Lys | Asn Leu Lys Asp Arg |
| Glu Met Ala Val Phe | Glu Gly Gly Lys Val | Leu Lys Asp Arg Tyr |
| Met Ala Val Phe Gly | Gly Gly Lys Val Thr | Lys Asp Arg Tyr Gly |
| Ala Val Phe Gly Ala | Gly Lys Val Thr Ala | Asp Arg Tyr Gly Ser |
| Val Phe Gly Ala Ala | Lys Val Thr Ala Glu | Arg Tyr Gly Ser Trp |
| Phe Gly Ala Ala Ala | Val Thr Ala Glu Thr | Tyr Gly Ser Trp Met |
| Gly Ala Ala Ala Pro | Thr Ala Glu Thr Glu | Gly Ser Trp Met Ile |
| Ala Ala Ala Pro Tyr | Ala Glu Thr Glu Tyr | Ser Trp Met Ile Tyr |
| Ala Ala Pro Tyr Leu | Glu Thr Glu Tyr Gly | Trp Met Ile Tyr Thr |
| Ala Pro Tyr Leu Arg | Thr Glu Tyr Gly Lys | Met Ile Tyr Thr Tyr |
| Pro Tyr Leu Arg Lys | Glu Tyr Gly Lys Thr | Ile Tyr Thr Tyr Ser |
| Tyr Leu Arg Lys Ser | Tyr Gly Lys Thr Val | Tyr Thr Tyr Ser Gly |
| Leu Arg Lys Ser Glu | Gly Lys Thr Val Thr | Thr Tyr Ser Gly Leu |
| Arg Lys Ser Glu Lys | Lys Thr Val Thr Val | Tyr Ser Gly Leu Phe |
| Lys Ser Glu Lys Glu | Thr Val Thr Val Lys | Ser Gly Leu Phe Cys |
| Ser Glu Lys Glu Arg | Val Thr Val Lys Glu | Gly Leu Phe Cys Val |
| Glu Lys Glu Arg Leu | Thr Val Lys Glu Asp | Leu Phe Cys Val Thr |
| Lys Glu Arg Leu Glu | Val Lys Glu Asp Gln | Phe Cys Val Thr Val |
| Glu Arg Leu Glu Ala | Lys Glu Asp Gln Val | Cys Val Thr Val Asn |
| Arg Leu Glu Ala Gln | Glu Asp Gln Val Met | Val Thr Val Asn Pro |
| Leu Glu Ala Gln Thr | Asp Gln Val Met Gln | Thr Val Asn Pro Tyr |
| Glu Ala Gln Thr Arg | Gln Val Met Gln Gln | Val Asn Pro Tyr Lys |
| Ala Gln Thr Arg Pro | Val Met Gln Gln Asn | Asn Pro Tyr Lys Trp |
| Gln Thr Arg Pro Phe | Met Gln Gln Asn Pro | Pro Tyr Lys Trp Leu |
| Thr Arg Pro Phe Asp | Gln Gln Asn Pro Pro | Tyr Lys Trp Leu Pro |
| Arg Pro Phe Asp Leu | Gln Asn Pro Pro Lys | Lys Trp Leu Pro Val |
| Pro Phe Asp Leu Lys | Asn Pro Pro Lys Phe | Trp Leu Pro Val Tyr |
| Phe Asp Leu Lys Lys | Pro Pro Lys Phe Asp | Leu Pro Val Tyr Thr |
| Asp Leu Lys Lys Asp | Pro Lys Phe Asp Lys | Pro Val Tyr Thr Pro |
| Leu Lys Lys Asp Val | Lys Phe Asp Lys Ile | Val Tyr Thr Pro Glu |
| Lys Lys Asp Val Phe | Phe Asp Lys Ile Glu | Tyr Thr Pro Glu Val |
| Lys Asp Val Phe Val | Asp Lys Ile Glu Asp | Thr Pro Glu Val Val |
| Asp Val Phe Val Pro | Lys Ile Glu Asp Met | Pro Glu Val Val Ala |
| Val Phe Val Pro Asp | Ile Glu Asp Met Ala | Glu Val Val Ala Ala |
| Phe Val Pro Asp Asp | Glu Asp Met Ala Met | Val Val Ala Ala Tyr |
| Val Pro Asp Asp Lys | Asp Met Ala Met Leu | Val Ala Ala Tyr Arg |
| Pro Asp Asp Lys Gln | Met Ala Met Leu Thr | Ala Ala Tyr Arg Gly |
| Asp Asp Lys Gln Glu | Ala Met Leu Thr Phe | Ala Tyr Arg Gly Lys |
| Asp Lys Gln Glu Phe | Met Leu Thr Phe Leu | Tyr Arg Gly Lys Lys |
| Lys Gln Glu Phe Val | Leu Thr Phe Leu His | Arg Gly Lys Lys Arg |
| Gln Glu Phe Val Lys | Thr Phe Leu His Glu | Gly Lys Lys Arg Ser |
| Glu Phe Val Lys Ala | Phe Leu His Glu Pro | Lys Lys Arg Ser Glu |
| Phe Val Lys Ala Lys | Leu His Glu Pro Ala | Lys Arg Ser Glu Ala |
| Val Lys Ala Lys Ile | His Glu Pro Ala Val | Arg Ser Glu Ala Pro |
| Lys Ala Lys Ile Val | Glu Pro Ala Val Leu | Ser Glu Ala Pro Pro |
| Ala Lys Ile Val Ser | Pro Ala Val Leu Tyr | Glu Ala Pro Pro His |
| Lys Ile Val Ser Arg | Ala Val Leu Tyr Asn | Ala Pro Pro His Ile |
| Ile Val Ser Arg Glu | Val Leu Tyr Asn Leu | Pro Pro His Ile Phe |
| Pro His Ile Phe Ser | Arg Ser Lys Lys Asp | Gly Lys Leu Ala Ser |
| His Ile Phe Ser Ile | Ser Lys Lys Asp Gln | Lys Leu Ala Ser Ala |
| Ile Phe Ser Ile Ser | Lys Lys Asp Gln Ser | Leu Ala Ser Ala Asp |
| Phe Ser Ile Ser Asp | Lys Asp Gln Ser Pro | Ala Ser Ala Asp Ile |
| Ser Ile Ser Asp Asn | Asp Gln Ser Pro Gly | Ser Ala Asp Ile Glu |
| Ile Ser Asp Asn Ala | Gln Ser Pro Gly Lys | Ala Asp Ile Glu Thr |
| Ser Asp Asn Ala Tyr | Ser Pro Gly Lys Gly | Asp Ile Glu Thr Tyr |
| Asp Asn Ala Tyr Gln | Pro Gly Lys Gly Thr | Ile Glu Thr Tyr Leu |
| Asn Ala Tyr Gln Tyr | Gly Lys Gly Thr Leu | Glu Thr Tyr Leu Leu |
| Ala Tyr Gln Tyr Met | Lys Gly Thr Leu Glu | Thr Tyr Leu Leu Glu |
| Tyr Gln Tyr Met Leu | Gly Thr Leu Glu Asp | Tyr Leu Leu Glu Lys |
| Gln Tyr Met Leu Thr | Thr Leu Glu Asp Gln | Leu Leu Glu Lys Ser |
| Tyr Met Leu Thr Asp | Leu Glu Asp Gln Ile | Leu Glu Lys Ser Arg |
| Met Leu Thr Asp Arg | Glu Asp Gln Ile Ile | Glu Lys Ser Arg Val |
| Leu Thr Asp Arg Glu | Asp Gln Ile Ile Gln | Lys Ser Arg Val Ile |
| Thr Asp Arg Glu Asn | Gln Ile Ile Gln Ala | Ser Arg Val Ile Phe |
| Asp Arg Glu Asn Gln | Ile Ile Gln Ala Asn | Arg Val Ile Phe Gln |
| Arg Glu Asn Gln Ser | Ile Gln Ala Asn Pro | Val Ile Phe Gln Leu |
| Glu Asn Gln Ser Ile | Gln Ala Asn Pro Ala | Ile Phe Gln Leu Lys |
| Asn Gln Ser Ile Leu | Ala Asn Pro Ala Leu | Phe Gln Leu Lys Ala |
| Gln Ser Ile Leu Ile | Asn Pro Ala Leu Glu | Gln Leu Lys Ala Glu |
| Ser Ile Leu Ile Thr | Pro Ala Leu Glu Ala | Leu Lys Ala Glu Arg |
| Ile Leu Ile Thr Gly | Ala Leu Glu Ala Phe | Lys Ala Glu Arg Asp |
| Leu Ile Thr Gly Glu | Leu Glu Ala Phe Gly | Ala Glu Arg Asp Tyr |
| Ile Thr Gly Glu Ser | Glu Ala Phe Gly Asn | Glu Arg Asp Tyr His |
| Thr Gly Glu Ser Gly | Ala Phe Gly Asn Ala | Arg Asp Tyr His Ile |
| Gly Glu Ser Gly Ala | Phe Gly Asn Ala Lys | Asp Tyr His Ile Phe |
| Glu Ser Gly Ala Gly | Gly Asn Ala Lys Thr | Tyr His Ile Phe Tyr |
| Ser Gly Ala Gly Lys | Asn Ala Lys Thr Val | His Ile Phe Tyr Gln |
| Gly Ala Gly Lys Thr | Ala Lys Thr Val Arg | Ile Phe Tyr Gln Ile |
| Ala Gly Lys Thr Val | Lys Thr Val Arg Asn | |
| Gly Lys Thr Val Asn | Thr Val Arg Asn Asp | |
| Lys Thr Val Asn Thr | Val Arg Asn Asp Asn | |
| Thr Val Asn Thr Lys | Arg Asn Asp Asn Ser | |
| Val Asn Thr Lys Arg | Asn Asp Asn Ser Ser | |
| Asn Thr Lys Arg Val | Asp Asn Ser Ser Arg | |
| Thr Lys Arg Val Ile | Asn Ser Ser Arg Phe | |
| Lys Arg Val Ile Gln | Ser Ser Arg Phe Gly | |
| Arg Val Ile Gln Tyr | Ser Arg Phe Gly Lys | |
| Val Ile Gln Tyr Phe | Arg Phe Gly Lys Phe | |
| Ile Gln Tyr Phe Ala | Phe Gly Lys Phe Ile | |
| Gln Tyr Phe Ala Val | Gly Lys Phe Ile Arg | |
| Tyr Phe Ala Val Ile | Lys Phe Ile Arg Ile | |
| Phe Ala Val Ile Ala | Phe Ile Arg Ile His | |
| Ala Val Ile Ala Ala | Ile Arg Ile His Phe | |
| Val Ile Ala Ala Ile | Arg Ile His Phe Gly | |
| Ile Ala Ala Ile Gly | Ile His Phe Gly Ala | |
| Ala Ala Ile Gly Asp | His Phe Gly Ala Thr | |
| Ala Ile Gly Asp Arg | Phe Gly Ala Thr Gly | |
| Ile Gly Asp Arg Ser | Gly Ala Thr Gly Lys | |
| Gly Asp Arg Ser Lys | Ala Thr Gly Lys Leu | |
| Asp Arg Ser Lys Lys | Thr Gly Lys Leu Ala | |

Any one of the peptide reagents optionally can be modified at either or both of the N-terminal and C-terminal ends. N-terminal modifications include for example: acetylation [Ac], benzyloxycarbonyl [Cbz], biotin [Btn], cinnamoylation [Cinn], dabcyl [Dabc], dabsyl [Dabs], innamoylation [Cinn], dabcyl [Dabc], dabsyl [Dabs], dansyl [Dans], dinitrophenyl [Dnp], fluorescein [Flc], FMOC [Fmoc], formylation [Form], lissamine rhodamine [Liss], myristoylation [Mgrs], N-methyl [Nme], palmitoylation [Palm], steroylation [Ster], and 7-methoxycoumarin acetic acid [Mca]. C-terminal modifications include for example: amide [NH2], 4-Branch MAP resin [MAPC], and hydroxyl [OH].

Given a protein and thus a starting amino acid sequence from which a peptide reagent is to be derived, the peptide, or a library of multiple peptides, including peptides with modifications to either or both terminal ends, can be prepared by readily commercially accessible custom peptide synthesis services. Such services are now routinely available from, for example Sigma-Genosys (as PEPscreen®), Invitrogen and GeneTel Laboratories.

Peptide reagents according to the present disclosure can be tested for inhibition of autoantibody binding to the target protein by any of several detection methods as will be recognized by those of skill in the art. Typically a peptide reagent is prepared in a diluent to produce several solutions of varying concentrations. Each solution is combined with a selected amount of a test sample containing a known amount of autoantibody and target protein. A detection conjugate that includes a detectable label and a specific binding partner, i.e. antibody, against the target protein is also added. A signal generated by the detection conjugate can be used to quantify the relative inhibitory activity of each dilution of the peptide reagent with respect to autoantibody binding to the target protein.

For example, equimolar starting solutions of each peptide reagent, each having a different amino acid sequence derived from the target protein, can be obtained and then diluted in a suitable pre-incubation diluent to give solutions of pre-selected, varying concentrations, typically in the nmol/mL range. The target protein, typically a recombinant protein, can be coated in a suitable buffer solution on a microplate and maintained under conditions sufficient to obtain binding of the target protein to the plate, for example at 38° C., for about 1 h. The protein can then be overcoated sequentially with bovine serum albumin and a solution of sucrose in PBS. A detection conjugate can be prepared by labeling a murine anti-human IgG with a detectable label according to labeling methods well-known in the art. For example, the detectable label can be but is not limited to a chemiluminescent compound, such s an acridinium compound.

Each dilution of the inhibitor peptide reagent is then mixed, preferably at about a 1:1 ratio by volume, with a test sample that contains a known amount of endogenous autoantibodies to the target self-antigen. The resulting solutions are arrayed in microplates, sealed and maintained under conditions sufficient to obtain binding of the peptide reagent to the autoantibodies, for example for a period of about 6 to 24 hours at ambient temperature. Test samples that are positive and low controls are diluted with a suitable preincubation diluent and arrayed, for example in triplicate, on the microplate. The plates are incubated under conditions sufficient to obtain binding, for example at 37° C. for at least about 2 hours, and the plate is washed with a suitable buffer such as ARCHITECT® Wash Buffer. A detection conjugate is then added to the plate. For example, a detection conjugate can be a murine anti-human IgG specific monoclonal antibody conjugated to a detectable label. The plate is incubated again under conditions sufficient to achieve binding of the detection conjugate to the target self-antigen, for example at 37° C. for about 1 hour, before a final wash with the wash buffer.

For detection, the microplate is processed according to methods appropriate for the particular label and detection method selected. For example, when using a detection conjugate in which an acridinium compound is the detectable label, the microplate is loaded into a microplate reader (e.g. a Mithras microplate reader, Berthold Technologies Inc, Oak Ridge, Tenn.), and then equilibrated at a suitable temperature, for example at 28° C. A chemiluminescence signal from each well is recorded for a period of seconds following sequential addition of a pre-trigger solution and a trigger solution. The resulting chemiluminescent signals are then recorded. Data analysis of the signals can include a comparison of the signals as a plot of the ratio of signal to the low control (S/LC) against concentration of each peptide reagent to reveal the relative strength of inhibition by each peptide reagent.

### C. Immunoassay for Detecting a Protein of Interest in a Test Sample

The present disclosure also relates methods of using the peptide reagents as disclosed herein in immunoassays for detecting protein analytes of interest in a test sample in which autoantibodies against the target protein may or may not be present. The protein analytes of interest are typically self-antigens. As set forth elsewhere herein, examples of self-antigens which are proteins for which autoantibodies have been described include but are not limited to cardiac troponin, myeloperoxidase (MPO), prostate specific antigen (PSA), and thyroid stimulating hormone (TSH). It will be understood that the peptide reagents and related methods described herein are also applicable to the detection of any other protein of diagnostic interest for which autoantibodies not yet described may interfere with immunodetection of the protein.

The methods of the present disclosure involves obtaining a test sample from a subject and then detecting the presence of a protein of interest, especially a self-antigen of clinical interest, using immunodetection, while compensating for the presence of any autoantibodies against the analyte that may be present in the sample. This is achieved in part by providing a peptide reagent derived from the protein, which inhibits binding to the protein of the autoantibody that may be present in the sample.

### Immunoassay Methods

It will be recognized that methods of the present disclosure can be applied to immunoassays carried out in any of a wide variety of formats. The various immunoassay formats can be applied both to detection per se of a protein of interest, and also to testing of peptide reagents as disclosed herein to evaluate the inhibitory strength of a peptide reagent. A general review of immunoassays is available in METHODS IN CELL BIOLOGY VOLUME 37: ANTIBODIES IN CELL BIOLOGY, Asai, ed. Academic Press, Inc. New York (1993), and BASIC AND CLINICAL IMMUNOLOGY 7TH EDITION, Stites & Terr, eds. (1991), which are herein incorporated by reference in their entirety.

A peptide reagent according to the present disclosure assists in immunodetection of at least one protein (antigen) of interest in a test sample in which autoantibodies to the protein may be present. As described elsewhere herein, the protein from which the peptide reagent is derived can be, for example, selected from the group consisting of: cardiac troponin I, cardiac troponin T, thyroid stimulating hormone (TSH), beta-human chorionic gonadotropin (beta-HCG), myeloperoxidase (MPO), prostate specific antigen (PSA), human B-type natriuretic peptide (hBNP), myosin light chain 2, myosin-6 and myosin-7. Typically the test sample is for example whole blood, serum or plasma, but can be any biological material, preferably is a biological fluid, suspected of containing a protein of interest and which may also include autoantibodies to the protein of interest.

In use, at least one peptide reagent as disclosed herein is combined with the test sample to form a first mixture. Thus the first mixture contains at least the peptide reagent, and may contain an amount of the target protein and any autoantibodies against the target protein. When the target protein and endogenous autoantibodies against the protein are present in the sample, the peptide reagent disrupts, i.e. blocks the interaction between the autoantibody in the test sample and the protein, leaving the target protein free for specific binding with another binding partner. The method then proceeds according to a typical sandwich immunoassay format. For example, a second mixture is then prepared by combining the first mixture and a first specific binding partner, namely an antibody that binds specifically with the protein of interest. The protein and antibody pair form a first specific binding partner-protein complex. A detection conjugate, i.e. an antibody conjugated to a detectable label, is then introduced to the second mixture. The antibody of the detection conjugate is also a specific binding partner of the protein, i.e. a second specific binding partner. The antibody of the detection conjugate binds to the first specific binding partner-protein complex to form an immunodetection complex that includes the first specific binding partner, protein and second specific binding partner. As the peptide reagent prevents binding of any autoantibody present in the sample to the target protein, the peptide reagent thus prevents autoantibodies from interfering with formation of the immunodetection complex. A signal is generated by or emitted from the detectable label on the detection conjugate, and the signal is used to detect presence of the protein of interest in the test sample. The signal generated by the detection conjugate is proportional to the concentration of the protein of interest as determined by the rate of formation (k1) of the immunodetection complex versus the rate of dissociation of the immunodetection complex (k2).

The method may involve, for example, use of an acridinium compound as the detectable label. When an acridinium compound is used, the method may further include generating or providing a source of hydrogen peroxide to the second mixture, adding a basic solution to the resulting mixture, and measuring the light signal generated or emitted and detecting the protein of interest in the sample. The hydrogen peroxide source may be a buffer, a solution containing hydrogen peroxide, or a hydrogen peroxide generating enzyme. The basic solution is for example a solution having a pH of at least about 10.

The method can optionally involve use of a solid phase. For example, the first specific binding partner can be immobilized on a solid phase either before or after the formation of the first specific binding partner-protein complex. The second specific binding partner can be immobilized on a solid phase either before or after formation of the first specific binding partner-protein-second specific binding partner complex. The solid phase when used can be any suitable material with sufficient surface affinity to bind the antibodies being used, and can take any of a number of forms, such as a magnetic particle, bead, test tube, microtiter plate, cuvette, membrane, a scaffolding molecule, quartz crystal, film, filter paper, disc or a chip. Useful solid phase materials include: natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, tubes, particulates, or plates, or they may be coated onto, bonded, or laminated to appropriate inert carriers, such as paper, glass, plastic films, fabrics, or the like. Nitrocellulose has excellent absorption and adsorption qualities for a wide variety of reagents including monoclonal antibodies. Nylon also possesses similar characteristics and also is suitable.

Alternatively, the solid phase can constitute microparticles. Microparticles useful in the present disclosure can be selected by one skilled in the art from any suitable type of particulate material and include those composed of polystyrene, polymethylacrylate, polypropylene, latex, polytetrafluoroethylene, polyacrylonitrile, polycarbonate, or similar materials. Further, the microparticles can be magnetic or paramagnetic microparticles, such as carboxylated magnetic microparticles. The methods of the present disclosure can be adapted for use in systems that utilize microparticle technology including automated and semi-automated systems wherein the solid phase comprises a microparticle. Such systems include those described in pending U.S. Pat. No. 425,651 and U.S. Pat. No. 5,089,424, which correspond to published EPO App. Nos. EP 0 425 633 and EP 0 424 634, respectively, and U.S. Pat. No. 5,006,309.

In particular embodiments, the solid phase includes one or more electrodes. Antibodies can be affixed, directly or indirectly, to the electrode(s). In one embodiment, for example, an antibody of the first specific binding partner can be affixed to magnetic or paramagnetic microparticles, which are then positioned in the vicinity of the electrode surface using a magnet. Systems in which one or more electrodes serve as the solid phase are useful where detection is based on electrochemical interactions. Exemplary systems of this type are described, for example, in U.S. Pat. No. 6,887,714 (issued May 3, 2005). The basic method is described further below with respect to electrochemical detection.

Other considerations affecting the choice of a solid phase include the ability to minimize non-specific binding of labeled entities and compatibility with the labeling system employed. For, example, solid phases used with fluorescent labels should have sufficiently low background fluorescence to allow signal detection.

Thus, according to the present disclosure, an immunoassay of the present disclosure to detect the presence of a protein of interest is a heterogeneous assay employing a solid phase which can be a solid support. The immunoassay can be performed for example by immobilizing an exogenous antibody on the solid phase, wherein the exogenous antibody is reactive with at least one epitope on the protein of interest and functions as the first specific binding partner. The peptide reagent is introduced to the test sample. The test sample is then contacted with first specific binding partner, under conditions sufficient for specific binding of the first specific binding partner to the protein of interest, thus forming a first specific binding partner-protein complex bound to the solid phase. In the case of a test sample containing at least one autoantibody against the protein, the peptide reagent blocks the interaction between the protein of interest and the autoantibody. The first specific binding partner-protein complex bound to the solid phase is contacted with the detection conjugate under conditions sufficient for specific binding of the detection conjugate to any of the protein of interest that is present in the test sample. An immunodetection complex is thus formed, which includes the first specific binding partner-protein complex and the detection conjugate.

Typically the detection conjugate includes a detectable label. Depending on the detection approach used, an optical, electrical, or change-of-state signal of the immunodetection complex is measured. The immunodetection complex is thus typically a configuration of molecules that once formed generates a signal susceptible to physical detection and/or quantification. Although the immunoassay is described above as including a sequence of steps for illustrative purposes, the test sample may be contacted with the first (capture) antibody and the second (detection) antibody simultaneously or sequentially, in any order. Regardless of the order of contact, if autoantibodies are present in the sample, the peptide reagent blocks interaction of the protein of interest with the autoantibodies that are present in the test sample.

In one format of a sandwich immunoassay according to the present disclosure, detecting comprises detecting a signal from the solid phase-affixed immunodetection complex, which includes the first specific binding partner, protein of interest and second specific binding partner (detection conjugate). In one embodiment, the immunodetection complex is separated from the solid phase, typically by washing, and the signal from the bound label is detected. In another format of a sandwich immunoassay according to the present disclosure, the immunodetection complex remains a solid phase-affixed complex, which is then detected.

### Antibodies

In the immunoassays according to the present disclosure, the first specific binding partner can be an antibody including a polyclonal antibody, a monoclonal antibody, a chimeric antibody, a human antibody, an affinity maturated antibody or an antibody fragment. Similarly, the second antibody can be a polyclonal antibody, a monoclonal antibody, a chimeric antibody, a human antibody, an affinity maturated antibody or an antibody fragment.

While monoclonal antibodies are highly specific to the protein/antigen, a polyclonal antibody can preferably be used as the capture (first) antibody to immobilize as much of the protein/antigen as possible. A monoclonal antibody with inherently higher binding specificity for the protein/antigen may then preferably be used as the detection (second) antibody. In any case, the antibody serving as the first specific binding partner and that serving as the second specific binding partner preferably recognize two non-overlapping epitopes on the protein to avoid blockage of, or interference by one with the epitope recognized by the other. Preferably the antibodies being used are capable of binding simultaneously to different epitopes on the protein of interest, each without interfering with the binding of the other.

Polyclonal antibodies are raised by injecting (e.g., subcutaneous or intramuscular injection) an immunogen into a suitable non-human mammal (e.g., a mouse or a rabbit). Generally, the immunogen should induce production of high titers of antibody with relatively high affinity for the target antigen (protein of interest).

If desired, the antigen may be conjugated to a carrier protein by conjugation techniques that are well known in the art. Commonly used carriers include keyhole limpet hemocyanin (KLH), thyroglobulin, bovine serum albumin (BSA), and tetanus toxoid. The conjugate is then used to immunize the animal.

The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections,” Langone, et al. eds. (Acad. Press, 1981)). Polyclonal antibodies produced by the animals can be further purified, for example, by binding to and elution from a matrix to which the target antigen is bound. Those of skill in the art will know of various techniques common in the immunology arts for purification and/or concentration of polyclonal, as well as monoclonal, antibodies (see, e.g., Coligan, et al. (1991) Unit 9, Current Protocols in Immunology, Wiley Interscience).

For many applications, monoclonal antibodies (mAbs) are preferred. The general method used for production of hybridomas secreting mAbs is well known (Kohler and Milstein (1975) Nature, 256:495). Briefly, as described by Kohler and Milstein, the technique involves isolating lymphocytes from regional draining lymph nodes of five separate cancer patients with either melanoma, teratocarcinoma or cancer of the cervix, glioma or lung, pooling the cells, and fusing the cells with SHFP-1. Hybridomas are screened for production of antibody that binds to cancer cell lines. Confirmation of specificity among mAbs can be accomplished using routine screening techniques such as ELISA to determine the elementary reaction pattern of the mAb of interest.

As used herein, the term “antibody” encompasses antigen-binding antibody fragments, e.g., single chain antibodies (scFv or others), which can be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage-bearing antigen-binding antibody fragments can be separated from non-binding phage by antigen affinity chromatography (McCafferty et al. (1990) Nature, 348: 552-554). Depending on the affinity of the antibody fragment, enrichment factors of 20-fold-1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus, even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment, natural VH and VL repertoires present in human peripheral blood lymphocytes are isolated from unimmunized donors by PCR. The V-gene repertoires can be spliced together at random using PCR to create a scFv gene repertoire which can be cloned into a phage vector to create a library of 30 million phage antibodies (Id.). From a single “naive” phage antibody library, binding antibody fragments have been isolated against more than 17 different antigens, including haptens, polysaccharides, and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor, and CEA (Griffiths et al. (1993) EMBO J. 12: 725-734). The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be prepared by any of a number of commercial services (e.g., Berkeley Antibody Laboratories, Bethyl Laboratories, Anawa, Eurogenetec, etc.).

### Detection Systems in General

As discussed above, immunoassays according to the present disclosure employ a second specific binding partner that typically includes an antibody specific to the protein of interest. In certain embodiments, the second specific binding partner includes a detectable label conjugated to the antibody, and function as a detection conjugate.

Detectable labels suitable for use in the detection conjugate include any compound or composition having a moiety that is detectable by spectroscopic, photochemical, biochemical, immunochemical, electrical, optical, or chemical means. Such labels include, for example, a radioactive label, an enzymatic label, a chemiluminescent label, a fluorescence label, a thermometric label, and an immuno-polymerase chain reaction label.

Thus for example, in an immunoassay employing an optical signal, the optical signal is measured as a protein concentration dependent change in chemiluminescence, fluorescence, phosphorescence, electrochemiluminescence, ultraviolet absorption, visible absorption, infrared absorption, refraction, surface plasmon resonance. In an immunoassay employing an electrical signal, the electrical signal is measured as an protein concentration dependent change in current, resistance, potential, mass to charge ratio, or ion count. In an immunoassay employing a change-of-state signal, the change of state signal is measured as an protein concentration dependent change in size, solubility, mass, or resonance.

More specifically, the label can be for example an enzyme, oligonucleotide, nanoparticle chemiluminophore, fluorophore, fluorescence quencher, chemiluminescence quencher, or biotin. Useful labels according to the present disclosure include magnetic beads (e.g., Dynabeads™), fluorescent dyes (e.g., fluorescein, Texas Red, rhodamine, green fluorescent protein) and the like (see, e.g., Molecular Probes, Eugene, Oreg., USA), chemiluminescent compounds such as acridinium (e.g., acridinium-9-carboxamide), phenanthridinium, dioxetanes, luminol and the like, radiolabels (e.g., ³H, ¹²⁵I, ³⁵S, ¹⁴C, or ³²P), catalysts such as enzymes (e.g., horse radish peroxidase, alkaline phosphatase, beta-galactosidase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label can be attached to the detection antibody to form the detection conjugate prior to, or during, or after contact with the biological sample. So-called “direct labels” are detectable labels that are directly attached to or incorporated into the detection antibody prior to use in the assay. Direct labels can be attached to or incorporated into the detection antibody by any of a number of means well known to those of skill in the art.

In contrast, so-called “indirect labels” typically bind to the detection antibody at some point during the assay. Often, the indirect label binds to a moiety that is attached to or incorporated into the detection agent prior to use. Thus, for example, a detection antibody can be biotinylated before use in an assay. During the assay, an avidin-conjugated fluorophore can bind the biotin-bearing detection agent, to provide a label that is easily detected.

In another example of indirect labeling, polypeptides capable of specifically binding immunoglobulin constant regions, such as polypeptide A or polypeptide G, can also be used as labels for detection antibodies. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542). Such polypeptides can thus be labeled and added to the assay mixture, where they will bind to the capture and detection antibodies, as well as to the autoantibodies, labeling all and providing a composite signal attributable to protein and autoantibody present in the sample.

Some labels useful in the present disclosure may require the use of an additional reagent(s) to produce a detectable signal. In an ELISA, for example, an enzyme label (e.g., beta-galactosidase) will require the addition of a substrate (e.g., X-gal) to produce a detectable signal. In immunoassay detection methods using an acridinium compound as a direct label, a basic solution and a source of hydrogen peroxide are added.

### Detection Systems—Exemplary Formats

Chemiluminescence Immunoassay: In an exemplary embodiment, a chemiluminescent compound is used in the above-described methods as a direct label as part of a detection conjugate. The chemiluminescent compound can be an acridinium compound. When an acridinium compound is used as the detectable label, then the above-described method may further include generating or providing a source of hydrogen peroxide to the mixture resulting from contacting the test sample with the first specific binding partner and the second specific binding partner (detection conjugate) and adding at least one basic solution to the mixture to generate a light signal. The light signal generated or emitted by the mixture is then measured to detect the protein of interest in the test sample.

The source of hydrogen peroxide may be a buffer solution or a solution containing hydrogen peroxide or an enzyme that generates hydrogen peroxide when added to the test sample. A hydrogen peroxide generating enzyme can be selected for example from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N6-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

The basic solution serves as a trigger solution, and the order in which the at least one basic solution and detectable label are added is not critical. The basic solution used in the method is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method described herein.

In a chemiluminescence immunoassay according to the present disclosure and using an acridinium compound as the detectable label, preferably the acridinium compound is an acridinium-9-carboxamide. Specifically, the acridinium-9-carboxamide has a structure according to formula I:

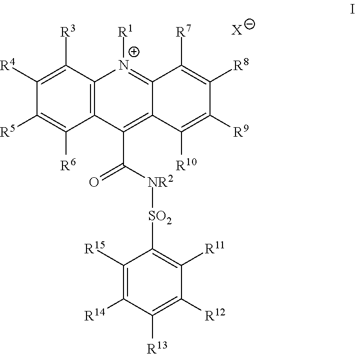

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

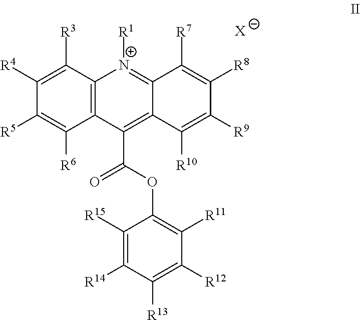

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Examples of acridinium-9-carboxylate aryl esters having the above formula II that can be used in the present disclosure include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

In addition to the at least one acridinium compound, the indicator solution can also contain at least one surfactant. Any surfactant that when dissolved in water, lowers the surface tension of the water and increases the solubility of organic compounds, can be used in the present invention. Examples of surfactants that can be used is one or more non-ionic or ionic surfactants (e.g., anionic, cationic or zwitterionic surfactants). Examples of non-ionic surfactants that can be used include, but are not limited to, t-octylpheoxypolyethoxyethanol (TRITON X-100, Sigma Aldrich, St. Louis, Mo.), polyoxyethylenesorbitan monolaurate (Tween 20), nonylphenol polyoxyethylene ether (Nonidet P10), decyldimethylphosphine oxide (APO-10), Cyclohexyl-n-ethyl-β-D-Maltoside, Cyclohexyl-n-hexyl-β-D-Maltoside, Cyclohexyl-n-methyl-β-D-Maltoside, n-Decanoylsucrose, n-Decyl-β-D-glucopyranoside, n-Decyl-β-D-maltopyranoside, n-Decyl-β-D-thiomaltoside, Digitonin, n-Dodecanoyl sucrose, n-Dodecyl-β-D-glucopyranoside, n-Dodecyl-β-D-maltoside, polyoxyethylene (10) dodecyl ether (Genapol C-100), isotridecanol polyglycol ether (Genapol X-80), isotridecanol polyglycol ether (Genapol X-100), Heptane-1,2,3-triol, n-Heptyl-β-D-glucopyranoside, n-Heptyl-β-D-thioglucopyranoside and combinations thereof. An example of a ionic surfactant that can be used include, sodium cholate, chenodeoxycholic acid, cholic acid, dehydrocholic acid, docusate sodium, docusate sodium salt, glycocholic acid hydrate, glycodeoxycholic acid monohydrate, glycolithocholic acid ethyl ester, N-lauroylsarcosine sodium salt, N-lauroylsarcosine, lithium dodecyl sulfate, calcium propionate, 1-octanesulfonic acid sodium salt, sodium 1-butanesulfonate, sodium chenodeoxycholate, sodium cholate hydrate, sodium 1-decanesulfonate, sodium 1-decanesulfonate, sodium deoxycholate, sodium deoxycholate monohydrate, sodium dodecylbenzenesulfonate, sodium dodecyl sulfate, sodium glycochenodeoxycholate, sodium glycocholate hydrate, sodium 1-heptanesulfonate, sodium hexanesulfonate, sodium 1-nonanesulfonate, sodium octyle sulfate, sodium pentanesulfonate, sodium 1-propanesulfonate hydrate, sodium taurodeoxycholate hydrate, sodium taurohyodeoxycholate hydrate, sodium tauroursodeoxycholate, taurocholic acid sodium salt hydrate, taurolithocholic acid 3-sulfate disodium salt, Triton® X-200, Triton® QS-15, Triton® QS-44, Triton® XQS-20, Trizma® dodecyl sulfate, ursodeoxycholic acid, alkyltrimethylammonium bromide, amprolium hydrocholoride, benzalkonium chloride, benzethonium hydroxide, benzyldimethylhexadecylammonium chloride, benzyldodecyldimethylammonium bromide, choline p-toluenesulfonate salt, dimethyldioctadecylammonium bromide, dodecylethyldimethylammonium bromide, dodecyltrimethylammonium bromide, ethylhexadecyldimethylammonium bromide, Ggirard's reagent, hexadecylpyridinium bromide, hexadecylpyridinium chloride monohydrate, hexadecylpyridinium chloride monohydrate, hexadecyltrimethylammonium bromide, hexadecyltrimethylammonium p-toluenesulfonate, hexadecyltrimethylammonium bromide, hexadecyltrimethylammonium p-toluenesulfonate, Hyamine® 1622, methylbenzethonium chloride, myristyltrimethylammonium bromide, oxyphenonium bromide, N,N′,N′-polyoxyethylene (10)-N-tallow-1,3-diaminopropane, tetraheptylammonium bromide, tetrakis(decyl)ammonium bromide, thonzonium bromide and Luviquat™ FC370, Luviquat™ HM 552, Luviquat™ HOLD, Luviquat™ MS 370, Luviquat™ PQ 11PN and combinations thereof (all available from Sigma Aldrich, St. Louis, Mo.).

Optionally, the test sample may be treated prior to the addition of any one or more of the at least one basic solution, hydrogen peroxide source and detectable label. Such treatment may include dilution, ultrafiltration, extraction, precipitation, dialysis, chromatography and digestion. Such treatment may be in addition to and separate from any pretreatment that the test sample may receive or be subjected to as discussed previously herein. Moreover, if such treatment methods are employed with respect to the test sample, such treatment methods are such that the protein of interest remains in the test sample at a concentration proportional to that in an untreated test sample (e.g., namely, a test sample that is not subjected to any such treatment method(s)).

As mentioned briefly previously herein, the time and order in which the test sample, the at least one basic solution, source of hydrogen peroxide and the detectable label are added to form a mixture is not critical. Additionally, the mixture formed by the at least one basic solution, hydrogen peroxide source and the detectable label, can optionally be allowed to incubate for a period of time. For example, the mixture can be allowed to incubate for a period of time of from about 1 second to about 60 minutes. Specifically, the mixture can be allowed to incubate for a period of from about 1 second to about 18 minutes.

When a chemiluminescent detectable label is used, after the addition of the at least one basic solution, hydrogen peroxide source, and the detectable label to the test sample, a detectable signal, namely, a chemiluminescent signal, is generated. The signal generated by the mixture is detected for a fixed duration of time. Preferably, the mixture is formed and the signal is detected concurrently. The duration of the detection may range from about 0.01 to about 360 seconds, more preferably from about 0.1 to about 30 seconds, and most preferably from about 0.5 to about 5 seconds. Chemiluminescent signals generated can be detected using routine techniques known to those skilled in the art.

Thus, in a chemiluminescent immunoassay according to the present disclosure, a chemiluminescent detectable label is used and added to the test sample, the chemiluminescent signal generated after the addition of the basic solution and the detectable label indicates the presence of the protein of interest in the test sample, which signal can be detected. The amount or concentration of the protein of interest in the test sample can be quantified based on the intensity of the signal generated. Specifically, the amount of the protein of interest contained in a test sample is proportional to the intensity of the signal generated. Specifically, the amount of the protein of interest present can be quantified based on comparing the amount of light generated to a standard curve for the protein of interest or by comparison to a reference standard. The standard curve can be generated using serial dilutions or solutions to the protein of interest of known concentration, by mass spectroscopy, gravimetrically and by other techniques known in the art.

Fluorescence Polarization Immunoassay (FPIA): In an exemplary embodiment, a fluorescent label is employed in a fluorescence polarization immunoassay (FPIA) according to the invention. Generally, fluorescent polarization techniques are based on the principle that a fluorescent label, when excited by plane-polarized light of a characteristic wavelength, will emit light at another characteristic wavelength (i.e., fluorescence) that retains a degree of the polarization relative to the incident light that is inversely related to the rate of rotation of the label in a given medium. As a consequence of this property, a label with constrained rotation, such as one bound to another solution component with a relatively lower rate of rotation, will retain a relatively greater degree of polarization of emitted light than when free in solution.

This technique can be employed in an immunoassay according to the present disclosure, for example, by selecting reagents such that binding of the fluorescently labeled entities forms a complex sufficiently different in size such that a change in the intensity light emitted in a given plane can be detected. For example, when a labeled cardiac troponin antibody, i.e. a second specific binding partner is bound by one or more cardiac troponin antigens bound to the first specific binding partner, the resulting complex is sufficiently larger, and its rotation is sufficiently constrained, relative to any free labeled cardiac troponin antibody that binding is easily detected.

Fluorophores useful in FPIA include fluorescein, aminofluorescein, carboxyfluorescein, and the like, preferably 5 and 6-aminomethylfluorescein, 5 and 6-aminofluorescein, 6-carboxyfluorescein, 5-carboxyfluorescein, thioureafluorescein, and methoxytriazinolyl-aminofluorescein, and similar fluorescent derivatives. Examples of commercially available automated instruments with which fluorescence polarization assays can be conducted include: the IMx system, the TDx system, and TDxFLx system (all available from Abbott Laboratories, Abbott Park, Ill.).

Scanning Probe Microscopy (SPM): The use of scanning probe microscopy (SPM) for immunoassays also is a technology to which the immunoassay methods of the present disclosure are easily adaptable. In SPM, in particular in atomic force microscopy, the capture antibody is affixed to the solid phase that in addition to being capable of binding autoantibodies, has a surface suitable for scanning. The capture antibody can, for example, be adsorbed to a plastic or metal surface. Alternatively, the capture antibody can be covalently attached to, e.g., derivatized plastic, metal, silicon, or glass according to methods known to those of ordinary skill in the art. Following attachment of the capture antibody, the test sample is contacted with the solid phase, and a scanning probe microscope is used to detect and quantify solid phase-affixed complexes. The use of SPM eliminates the need for labels that are typically employed in immunoassay systems. Such a system is described in U.S. Pat. No. 662,147, which is incorporated herein by reference.

MicroElectroMechanical Systems (MEMS): Immunoassays according to the present disclosure can also be carried out using a MicroElectroMechanical System (MEMS). MEMS are microscopic structures integrated onto silicon that combine mechanical, optical, and fluidic elements with electronics, allowing convenient detection of an protein of interest. An exemplary MEMS device suitable for use in the present disclosure is the Protiveris' multicantilever array. This array is based on chemo-mechanical actuation of specially designed silicon microcantilevers and subsequent optical detection of the microcantilever deflections. When coated on one side with a binding partner, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Electrochemical Detection Systems: In other embodiments, immunoassays according to the present disclosure are carried out using electrochemical detection, the techniques for which are well known to those skilled in the art. Such electrochemical detection often employs one or more electrodes connected to a device that measures and records an electrical current. Such techniques can be realized in a number of commercially available devices, such as the I-STAT® (Abbott Laboratories, Abbott Park, Ill.) system, which comprises a hand-held electrochemical detection instrument and self-contained assay-specific reagent cartridges. For example, in the present invention, the basic trigger solution could be contained in the self-contained hemoglobin reagent cartridge and upon addition of the test sample, a current would be generated at least one electrode that is proportional to the amount of hemoglobin in the test sample. A basic procedure for electrochemical detection has been described for example by Heineman and coworkers. This entailed immobilization of a primary antibody (Ab, rat-anti mouse IgG), followed by exposure to a sequence of solutions containing the antigen (Ag, mouse IgG), the secondary antibody conjugated to an enzyme label (AP-Ab, rat anti mouse IgG and alkaline phosphatase), and p-aminophenyl phosphate (PAPP). The AP converts PAPP to p-aminophenol (PAP_{R}, the “R” is intended to distinguish the reduced form from the oxidized form, PAP_{O}, the quinoneimine), which is electrochemically reversible at potentials that do not interfere with reduction of oxygen and water at pH 9.0, where AP exhibits optimum activity. PAP_{R} does not cause electrode fouling, unlike phenol whose precursor, phenylphosphate, is often used as the enzyme substrate. Although PAP_{R} undergoes air and light oxidation, these are easily prevented on small scales and short time frames. Picomole detection limits for PAP_{R} and femtogram detection limits for IgG achieved in microelectrochemical immunoassays using PAPP volumes ranging from 20 μl to 360 μL have been reported previously. In capillary immunoassays with electrochemical detection, the lowest detection limit reported thus far is 3000 molecules of mouse IgG using a volume of 70 μL and a 30 min or 25 min assay time.

In an exemplary embodiment employing electrochemical detection according to the present disclosure, an antibody serving as the first specific binding partner, which is reactive with the protein of interest, can be immobilized on the surface of an electrode, which is the solid phase. The electrode is then contacted with a test sample from, e.g., a human. Any protein in the sample binds to the first specific binding partner, e.g. antibody to form a solid phase-affixed complex. Autoantibodies present in the sample are blocked by the peptide reagent from interacting with the target protein and thus from interfering with binding of the target protein to the first specific binding partner. The solid phase-affixed complexes are contacted with the detection conjugate including a detectable label. Formation of an immunodetection complex that includes the first specific binding partner, protein, and detection conjugate results in generation of a signal by the detectable label, which is then detected.

Various electrochemical detection systems are described in U.S. Pat. No. 7,045,364 (issued May 16, 2006; incorporated herein by reference), U.S. Pat. No. 7,045,310 (issued May 16, 2006; incorporated herein by reference), U.S. Pat. No. 6,887,714 (issued May 3, 2005; incorporated herein by reference), U.S. Pat. No. 6,682,648 (issued Jan. 27, 2004; incorporated herein by reference); U.S. Pat. No. 6,670,115 (issued Dec. 30, 2003; incorporated herein by reference).

### D. Kits

The present disclosure also provides kits for assaying test samples for presence of an protein of interest wherein the test sample may contain autoantibodies. Kits according to the present disclosure include one or more reagents useful for practicing one or more immunoassays according to the present disclosure. A kit generally includes a package with one or more containers holding the reagents, as one or more separate compositions or, optionally, as admixture where the compatibility of the reagents will allow. The test kit can also include other material(s), which may be desirable from a user standpoint, such as a buffer(s), a diluent(s), a standard(s), and/or any other material useful in sample processing, washing, or conducting any other step of the assay.

In certain embodiments, a test kit for detecting and/or quantifying at least one protein of interest in a test sample includes a capture reagent comprising an antibody that binds to the protein of interest; and instructions for detecting and/or quantifying at least one protein of interest in a test sample. The kit may further include a conjugate which includes an antibody conjugated to a detectable label.

In certain embodiments, a test kit may include a humanized monoclonal antibody, wherein the humanized monoclonal antibody is specific for the protein of interest. This component can be used as a positive control in immunoassays according to the invention. If desired, this component can be included in the test kit in multiple concentrations to facilitate the generation of a standard curve to which the signal detected in the test sample can be compared. Alternatively, a standard curve can be generated by preparing dilutions of a single humanized monoclonal antibody solution provided in the kit.

Kits according to the present disclosure can include one or more peptide reagents having a sequence derived from the protein of interest, an antibody (first specific binding partner) that binds to at least one epitope on the protein of interest, a solid phase capable of binding the first specific binding partner, a second antibody that binds to at least one epitope on the protein of interest, and instructions for detecting or quantifying the protein of interest. In certain embodiments test kits according to the present disclosure may include the solid phase as a material such as a magnetic particle, a bead, a test tube, a microtiter plate, a cuvette, a membrane, a scaffolding molecule, a quartz crystal, a film, a filter paper, a disc or a chip.

Test kits according to the present disclosure can include for example non-human monoclonal antibodies against the protein of interest, as the first and second specific binding partners. The kit may also include a detectable label that can be or is conjugated to an antibody to provide a detection conjugate as the second specific binding partner.

In certain embodiments, the test kit includes the detectable label as at least one direct label, which may be an enzyme, oligonucleotide, nanoparticle chemiluminophore, fluorophore, fluorescence quencher, chemiluminescence quencher, or biotin. In some embodiments, the direct label is an acridinium compound such as an acridinium-9-carboxamide according to formula I:

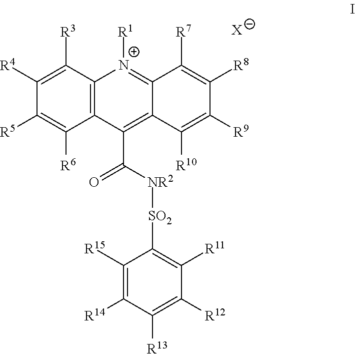

wherein R1 and R2 are each independently selected from the group consisting of: alkyl, alkenyl, alkynyl, aryl or aralkyl, sulfoalkyl, carboxyalkyl and oxoalkyl, and
wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X® is an anion.

Alternatively, the acridinium compound can be an acridinium-9-carboxylate aryl ester having a structure according to formula II:

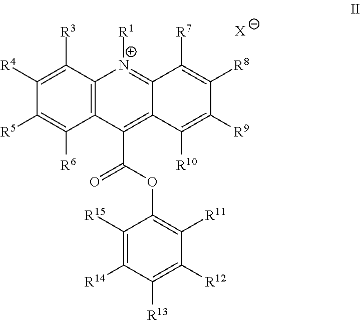

wherein R1 is an alkyl, alkenyl, alkynyl, aryl or aralkyl, sulfoalkyl, carboxyalkyl and oxoalkyl; and
wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

Test kits according to the present disclosure and which include an acridinium compound can also include a basic solution. For example, the basic solution can be a solution having a pH of at least about 10. In certain embodiments, test kits according to the present disclosure may further include a hydrogen peroxide source, such as a buffer solution, a solution containing hydrogen peroxide, or a hydrogen peroxide generating enzyme. For example, test kits may include an amount of a hydrogen peroxide generating enzymes selected from the following: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N6-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

In certain embodiments, test kits according to the present disclosure are configured for detection or quantification of one of the following specific analytes of interest cardiac troponin, thyroid stimulating hormone (TSH), beta human chorionic gonadotropin (beta-HCG); myeloperoxidase (MPO), prostate specific antigen (PSA), human B-type natriuretic peptide (BNP), myosin light chain 2, myosin-6 and myosin-7. In such embodiments, the test kits include at least one peptide reagent having a sequence derived from the protein of interest, a first antibody and a second antibody that each bind to an epitope on the selected protein of interest, i.e. a first antibody and a second antibody and second antibody that each bind to an epitope on one of the following: cardiac troponin, thyroid stimulating hormone (TSH), beta human chorionic gonadotropin (beta-HCG); myeloperoxidase (MPO), prostate specific antigen (PSA), human B-type natriuretic peptide (BNP), myosin light chain 2, myosin-6 and myosin-7.

Test kits according to the present disclosure preferably include instructions for carrying out one or more of the immunoassays of the invention. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### E. Adaptations of the Methods of the Present Disclosure

The present disclosure is for example applicable to the jointly owned commercial Abbott Point of Care (i-STAT™) electrochemical immunoassay system which performs sandwich immunoassays for several cardiac markers, including TnI, CKMB and BNP. Immunosensors and ways of operating them in single-use test devices are described in jointly owned Publication Nos. US 20030170881, US 20040018577, US 20050054078, and US 20060160164, each of which is incorporated herein by reference. Additional background on the manufacture of electrochemical and other types of immunosensors is found in jointly owned U.S. Pat. No. 5,063,081 which is also incorporated by reference.

By way of example, and not of limitation, examples of the present disclosures shall now be given.

### Example 1

### Inhibition of Anti-cTnI Autoantibody Binding to Cardiac Troponin-I (ELN Ref E000777-253)

Inhibitor working solutions: The peptides listed in Table 12 (obtained from Sigma-Genosys, PEPscreen custom library) were diluted in AxSYM® Troponin-I ADV Preincubation Diluent to give solutions ranging from 240 nmol/mL to 0 nmol/mL. An equimolar mixture of the peptides listed in Table 12 was prepared and diluted to give solutions ranging from 240 nmol/mL to 0 nmol/mL/.

TABLE 12
Peptide inhibitors of anti-cTnI autoantibody
binding to cardiac troponin-I

| | Amino- | | Carboxy- |
|---|---|---|---|
| Peptide# | terminus | Sequence | terminus |
| 1 | [Btn] | SSDAAREPRPAPAPI | [NH2] |
| 2 | [Btn] | VDEERYDIEAKVTKN | [NH2] |
| 3 | [Btn] | DIEAKVTKNITEIAD | [NH2] |
| 4 | [Btn] | LDLRAHLKQVKKEDT | [NH2] |
| 5 | [Btn] | ALSGMEGRKKKFES | [NH2] |

Microplate preparation: Recombinant human cardiac troponin-I (cTnI, BiosPacific, Emeryville, Calif.) was coated on white high-binding flat-bottom 96-well polystyrene microplates (Costar) in phosphate buffer (100 μL, 0.2 M, pH 8, 4 μg/mL) at 38° C., for 1 h, then overcoated sequentially with bovine serum albumin and 2% wt/v sucrose in PBS.

Chemiluminescent detection conjugate: A murine anti-human IgG (subtype IgG2b, kappa;) was labeled with a chemiluminescent acridinium-9-carboxamide. This antibody recognized all human IgG subtypes while having no significant reactivity toward human IgM or IgA, or rabbit, sheep or goat IgG.

Samples: A human serum sample containing a high level of endogenous antibodies to cardiac troponin-I was mixed 1:1 with each inhibitor dilution. The solutions were arrayed in a black polypropylene microplate, sealed and stored overnight at ambient temperature.

Assay protocol: The samples, positive and low controls (10 μL) were diluted with AxSYM® Troponin-I ADV Preincubation Diluent (90 μL) and arrayed in triplicate on the microplate. After incubating at 37° C. for 2 h, the plate was washed with ARCHITECT® Wash Buffer (6×, 350 μL). The murine anti-human IgG specific monoclonal-acridinium conjugate (100 μL) was then added and the plate incubated at 37° C. for 1 h, before a final wash with ARCHITECT® Wash Buffer (6×, 350 μL).

Chemiluminescent detection: The microplate was loaded into a Mithras microplate reader (Berthold Technologies Inc, Oak Ridge, Tenn.) equilibrated at 28° C. The chemiluminescence signal from each well was recorded for 2 s after the sequential addition of ARCHITECT® Pre-Trigger solution (100 μL) and ARCHITECT® Trigger solution (100 μL).

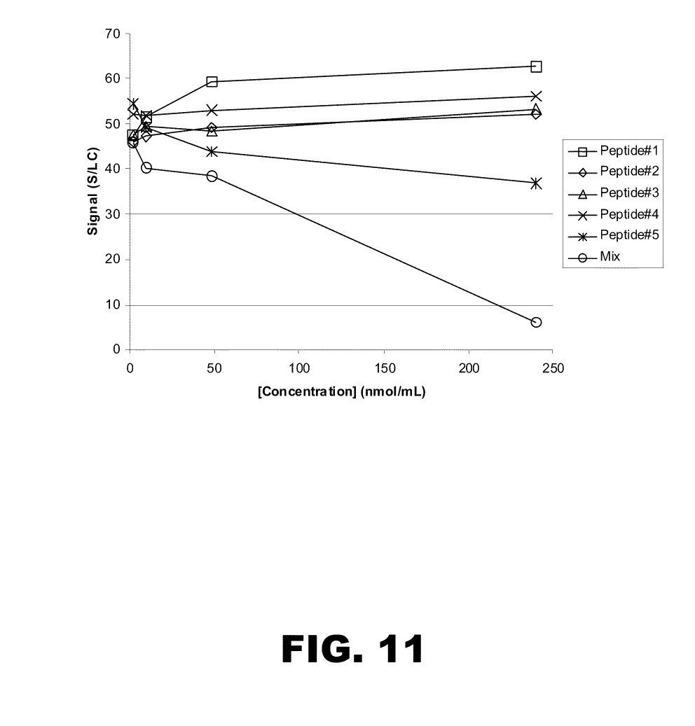

A plot of the ratio of signal to the low control (S/LC) (FIG. 11) showed that peptide #5 has the greatest inhibition of the binding of endogenous antibodies to the cTnI antigen on the microplate while the mixture of peptides gave a synergistic inhibitory effect.

### Example 2

### Inhibition of Anti-cTnI Autoantibody Binding to Cardiac Troponin-I (ELN Ref E000777-272)

The procedure of Example 1 was repeated using the peptides listed in Table 13.

TABLE 13
Peptide inhibitors of anti-cTnI autoantibody
binding to cardiac troponin-I

| | Amino- | | Carboxy- |
|---|---|---|---|
| Peptide# | terminus | Sequence | terminus |
| 1 | [Ac] | SSDAAREPRPAPAPI | [NH2] |
| 2 | [Ac] | VDEERYDIEAKVTKN | [NH2] |
| 3 | [[Ac] | DIEAKVTKNITEIAD | [NH2] |
| 4 | [Ac] | LDLRAHLKQVKKEDT | [NH2] |
| 5 | [[Ac] | ALSGMEGRKKKFES | [OH] |

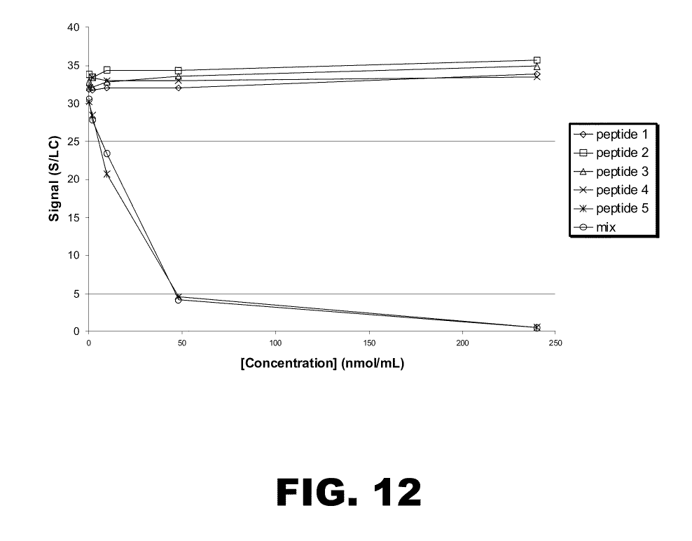

A plot of the ratio of signal to the low control (S/LC) (FIG. 12) showed that peptide #5 has the greatest inhibition of the binding of endogenous antibodies to the cTnI antigen on the microplate in this experiment, while the mixture of peptides again gave a synergistic inhibitory effect.

One skilled in the art would readily appreciate that the peptide reagents and related methods are well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The present disclosure illustratively described herein suitably may be practiced in the absence of any element or elements, limitation or limitations which are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A reagent for use in an immunoassay for determining the presence or amount of at least one protein in a test sample, the reagent comprising:
at least one peptide comprising at least 5 consecutive amino acid residues wherein the peptide is derived from said protein and further wherein said reagent is used to block the interaction between an endogenous antibody and said protein in the test sample.

2. The reagent of claim 1, wherein the protein is selected from the group consisting of: cardiac troponin I (SEQ ID NO:1), cardiac troponin T (SEQ ID NO:2), thyroid stimulating hormone (TSH) (SEQ ID NO:3), beta-human chorionic gonadotropin (beta-HCG) (SEQ ID NO:4), myeloperoxidase (MPO) (SEQ ID NO:5), prostate specific antigen (PSA) (SEQ ID NO:6), human B-type natriuretic peptide (hBNP) (SEQ ID NO:7), myosin light chain 2 (SEQ ID NO:8), myosin-6 (SEQ ID NO:9) and myosin-7 (SEQ ID NO:10).

3. The reagent of claim 1, wherein the peptide has a length of 5 consecutive amino acids to 15 consecutive amino acids.

4. The reagent of claim 1, wherein the protein is cardiac troponin I, and the peptide has a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

5. A reagent for use in an immunoassay for determining the presence or amount of at cardiac troponin I in a test sample, the reagent comprising a peptide having a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

6. A method of detecting at least one protein of interest in a test sample, the method comprising the steps of:
a. preparing a first mixture comprising a test sample suspected of containing at least one protein of interest and at least one reagent, wherein said reagent (1) is at least one peptide comprising at least 5 consecutive amino acid residues derived from said protein that binds to the antibody of interest; and (2) disrupts the interaction between an endogenous antibody in the test sample and the antigen;
b. preparing a second mixture comprising the first mixture and a first specific binding partner, wherein the first specific binding partner comprises an antibody, wherein the antibody binds with the protein of interest to form a first specific binding partner-protein complex; and
c. contacting the second mixture with a second specific binding partner, wherein the second specific binding partner comprises an antibody that has been conjugated to a detectable label and further wherein the second specific binding partner binds to the first specific binding partner-protein complex to form a first specific binding partner-protein-second specific binding partner complex; and
d. measuring the signal generated by or emitted from the detectable label and detecting the protein of interest in the test sample.

7. The method of claim 6, wherein the protein is selected from the group consisting of: cardiac troponin I, cardiac troponin T, thyroid stimulating hormone (TSH), beta-human chorionic gonadotropin (beta-HCG), myeloperoxidase (MPO), prostate specific antigen (PSA), human B-type natriuretic peptide (hBNP), myosin light chain 2, myosin-6 and myosin-7.

8. The method of claim 6, wherein the test sample is whole blood, serum or plasma.

9. The method of claim 6, wherein the first specific binding partner is immobilized to a solid phase either before or after the formation of the first specific binding partner-protein complex.

10. The method of claim 6, wherein the second specific binding partner is immobilized to a solid phase either before or after formation of the first specific binding partner-protein-second specific binding partner complex.

11. The method of claim 6, wherein the detectable label is selected from the group consisting of a radioactive label, an enzymatic label, a chemiluminescent label, a fluorescence label, a thermometric label, and an immuno-polymerase chain reaction label.

12. The method of claim 6, wherein said detectable label is an acridinium compound.

13. The method of claim 12 further comprising:
a. generating or providing a source of hydrogen peroxide to the second mixture contacted with a second specific binding partner;
b. adding a basic solution to the mixture of step (a);
c. measuring the light signal generated or emitted in step (b) and detecting the protein of interest in the sample.

14. The method of claim 12, wherein the acridinium compound is an acridinium-9-carboxamide having a structure according to formula I: 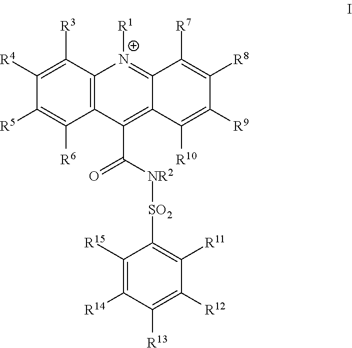
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and optionally, if present, X^{⊖} is an anion.

15. The method of claim 12, wherein the acridinium compound is an acridinium-9-carboxylate aryl ester having a structure according to formula II: 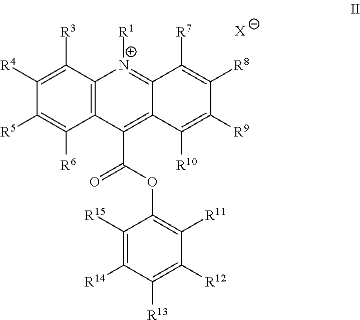
wherein R1 is an alkyl, alkenyl, alkynyl, aryl or aralkyl, sulfoalkyl, carboxyalkyl and oxoalkyl; and
wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and optionally, if present, XΘ is an anion.

16. The method of claim 6, wherein the reagent is a peptide having a length of 5 consecutive amino acids to 15 consecutive amino acids.

17. The method of claim 6, wherein the protein is cardiac troponin I, and the peptide has a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).

18. The method of claim 6, further comprising the step of quantifying the amount of protein of interest in the test sample by relating the amount of signal in step (d) to the amount of the one or more proteins of interest in the test sample either by use of a standard curve for the protein of interest or by comparison to a reference standard.

19. The method of claim 7, wherein the method is adapted for use in an automated system or semi-automated system.

20. A kit for detecting and/or quantifying at least one protein of interest in a test sample, the kit comprising: the reagent of claim 1; a capture reagent comprising an antibody that binds to the protein of interest; and instructions for detecting and/or quantifying at least one protein of interest in a test sample.

21. The kit of claim 20, wherein the kit further comprises a conjugate comprising an antibody conjugated to a detectable label.

22. The kit of claim 21, wherein the detectable label is selected from the group consisting of a radioactive label, an enzymatic label, a chemiluminescent label, a fluorescence label, a thermometric label, and an immuno-polymerase chain reaction label.

23. The kit of claim 22, wherein the detectable label is an acridinium compound.

24. The kit of claim 23, wherein the acridinium compound is an acridinium-9-carboxamide having a structure according to formula I: 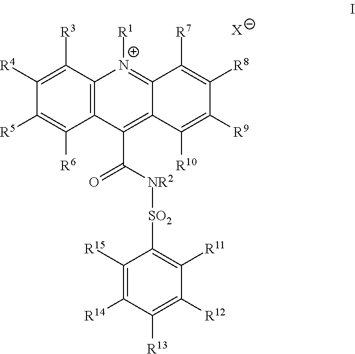
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

25. The kit of claim 23, wherein the acridinium compound is an acridinium-9-carboxylate aryl ester having a structure according to formula II: 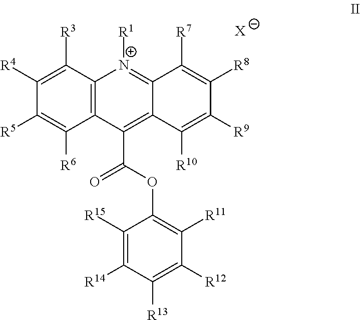
wherein R1 is an alkyl, alkenyl, alkynyl, aryl or aralkyl, sulfoalkyl, carboxyalkyl and oxoalkyl; and
wherein R3 through R15 are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

26. The kit of claim 23, further comprising a basic solution.

27. The kit of claim 26, wherein the basic solution is a solution having a pH of at least about 10.

28. The kit of claim 23, further comprising a hydrogen peroxide source.

29. The kit of claim 28, wherein the hydrogen peroxide source comprises a buffer or a solution containing hydrogen peroxide.

30. The kit of claim 28, wherein the hydrogen peroxide source comprises a hydrogen peroxide generating enzyme.

31. The kit of claim 30, wherein the hydrogen peroxide generating enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

32. The kit of claim 20, wherein the protein is cardiac troponin I, cardiac troponin T, thyroid stimulating hormone (TSH), beta-human chorionic gonadotropin (beta-HCG), myeloperoxidase (MPO), prostate specific antigen (PSA), human B-type natriuretic peptide (hBNP), myosin light chain 2, myosin-6 or myosin-7.

33. The kit of claim 20, wherein the reagent is a peptide having a length of 5 consecutive amino acids to 15 consecutive amino acids.

34. The kit of claim 33, wherein the protein is cardiac troponin I, and the peptide has a sequence comprising at least five consecutive amino acid residues from a sequence selected from the group consisting of SSDAAREPRPAPAPI (SEQ ID NO:11), VDEERYDIEAKVTKN (SEQ ID NO:12), DIEAKVTKNITEIAD (SEQ ID NO:13), LDLRAHLKQVKKEDT (SEQ ID NO:14), and ALSGMEGRKKKFES (SEQ ID NO:15).
